SEQUENCE LISTING (146 sequences)

SEQ ID NO: 1 - DNA, 15 bp, Artificial Sequence
  Synthetic Sequence
gctagacgttagcgt

SEQ ID NO: 2 - DNA, 15 bp, Artificial Sequence
  Synthetic Sequence
gctagatgttagcgt

SEQ ID NO: 3 - DNA, 15 bp, Artificial Sequence
  Synthetic Sequence
gctagacgttagcgt

SEQ ID NO: 4 - DNA, 15 bp, Artificial Sequence
  Synthetic Sequence
gctagacgttagcgt

SEQ ID NO: 5 - DNA, 15 bp, Artificial Sequence
  Synthetic Sequence
gcatgacgttgagct

SEQ ID NO: 6 - DNA, 20 bp, Artificial Sequence
  Synthetic Sequence
atggaaggtccagcgttctc

SEQ ID NO: 7 - DNA, 20 bp, Artificial Sequence
  Synthetic Sequence
atcgactctcgagcgttctc

SEQ ID NO: 8 - DNA, 20 bp, Artificial Sequence
  Synthetic Sequence
atcgactctcgagcgttctc

SEQ ID NO: 9 - DNA, 20 bp, Artificial Sequence
  Synthetic Sequence
atcgactctcgagcgttctc

SEQ ID NO: 10 - DNA, 20 bp, Artificial Sequence
  Synthetic Sequence
atggaaggtccaacgttctc

SEQ ID NO: 11 - DNA, 20 bp, Artificial Sequence
  Synthetic Sequence
gagaacgctggaccttccat

SEQ ID NO: 12 - DNA, 20 bp, Artificial Sequence
  Synthetic Sequence
gagaacgctcgaccttccat

SEQ ID NO: 13 - DNA, 20 bp, Artificial Sequence
  Synthetic Sequence
gagaacgctcgaccttcgat

SEQ ID NO: 14 - DNA, 20 bp, Artificial Sequence
  Synthetic Sequence
gagaacgctggaccttccat

SEQ ID NO: 15 - DNA, 20 bp, Artificial Sequence
  Synthetic Sequence
gagaacgatggaccttccat

SEQ ID NO: 16 - DNA, 20 bp, Artificial Sequence
  Synthetic Sequence
gagaacgctccagcactgat

SEQ ID NO: 17 - DNA, 20 bp, Artificial Sequence
  Synthetic Sequence
tccatgtcggtcctgatgct

SEQ ID NO: 18 - DNA, 20 bp, Artificial Sequence
  Synthetic Sequence
tccatgtcggtcctgatgct

SEQ ID NO: 19 - DNA, 20 bp, Artificial Sequence
  Synthetic Sequence
tccatgacgttcctgatgct

SEQ ID NO: 20 - DNA, 20 bp, Artificial Sequence
  Synthetic Sequence
tccatgtcggtcctgctgat

SEQ ID NO: 21 - DNA, 8 bp, Artificial Sequence
  Synthetic Sequence
tcaacgtt

SEQ ID NO: 22 - DNA, 8 bp, Artificial Sequence
  Synthetic Sequence
tcagcgct

SEQ ID NO: 23 - DNA, 8 bp, Artificial Sequence
  Synthetic Sequence
tcatcgat

SEQ ID NO: 24 - DNA, 8 bp, Artificial Sequence
  Synthetic Sequence
tcttcgaa

SEQ ID NO: 25 - DNA, 7 bp, Artificial Sequence
  Synthetic Sequence
caacgtt

SEQ ID NO: 26 - DNA, 8 bp, Artificial Sequence
  Synthetic Sequence
ccaacgtt

SEQ ID NO: 27 - DNA, 8 bp, Artificial Sequence
  Synthetic Sequence
aacgttct

SEQ ID NO: 28 - DNA, 8 bp, Artificial Sequence
  Synthetic Sequence
tcaacgtc

SEQ ID NO: 29 - DNA, 20 bp, Artificial Sequence
  Synthetic Sequence
atggactctccagcgttctc

SEQ ID NO: 30 - DNA, 20 bp, Artificial Sequence
  Synthetic Sequence
atggaaggtccaacgttctc

SEQ ID NO: 31 - DNA, 20 bp, Artificial Sequence
  Synthetic Sequence
atcgactctcgagcgttctc

SEQ ID NO: 32 - DNA, 20 bp, Artificial Sequence
  Synthetic Sequence
atggaggctccatcgttctc

SEQ ID NO: 33 - DNA, 20 bp, Artificial Sequence
  Synthetic Sequence
atcgactctcgagcgttctc

SEQ ID NO: 34 - DNA, 20 bp, Artificial Sequence
  Synthetic Sequence
atcgactctcgagcgttctc

SEQ ID NO: 35 - DNA, 20 bp, Artificial Sequence
  Synthetic Sequence
tccatgtcggtcctgatgct

SEQ ID NO: 36 - DNA, 20 bp, Artificial Sequence
  Synthetic Sequence
tccatgccggtcctgatgct

SEQ ID NO: 37 - DNA, 20 bp, Artificial Sequence
  Synthetic Sequence
tccatggcggtcctgatgct

SEQ ID NO: 38 - DNA, 20 bp, Artificial Sequence
  Synthetic Sequence
tccatgacggtcctgatgct

SEQ ID NO: 39 - DNA, 20 bp, Artificial Sequence
  Synthetic Sequence
tccatgtcgatcctgatgct

SEQ ID NO: 40 - DNA, 20 bp, Artificial Sequence
  Synthetic Sequence
tccatgtcgctcctgatgct

SEQ ID NO: 41 - DNA, 20 bp, Artificial Sequence
  Synthetic Sequence
tccatgtcgtccctgatgct

SEQ ID NO: 42 - DNA, 20 bp, Artificial Sequence
  Synthetic Sequence
tccatgacgtgcctgatgct

SEQ ID NO: 43 - DNA, 20 bp, Artificial Sequence
  Synthetic Sequence
tccataacgttcctgatgct

SEQ ID NO: 44 - DNA, 20 bp, Artificial Sequence
  Synthetic Sequence
tccatgacgtccctgatgct

SEQ ID NO: 45 - DNA, 20 bp, Artificial Sequence
  Synthetic Sequence
tccatcacgtgcctgatgct

SEQ ID NO: 46 - DNA, 19 bp, Artificial Sequence
  Synthetic Sequence
ggggtcaacgttgacgggg

SEQ ID NO: 47 - DNA, 19 bp, Artificial Sequence
  Synthetic Sequence
ggggtcagtcgtgacgggg

SEQ ID NO: 48 - DNA, 15 bp, Artificial Sequence
  Synthetic Sequence
gctagacgttagtgt

SEQ ID NO: 49 - DNA, 20 bp, Artificial Sequence
  Synthetic Sequence
tccatgtcgttcctgatgct

SEQ ID NO: 50 - DNA, 24 bp, Artificial Sequence
  Synthetic Sequence
accatggacgatctgtttcccctc

SEQ ID NO: 51 - DNA, 18 bp, Artificial Sequence
  Synthetic Sequence
tctcccagcgtgcgccat

SEQ ID NO: 52 - DNA, 24 bp, Artificial Sequence
  Synthetic Sequence
accatggacgaactgtttcccctc

SEQ ID NO: 53 - DNA, 24 bp, Artificial Sequence
  Synthetic Sequence
accatggacgagctgtttcccctc

SEQ ID NO: 54 - DNA, 24 bp, Artificial Sequence
  Synthetic Sequence
accatggacgacctgtttcccctc

SEQ ID NO: 55 - DNA, 24 bp, Artificial Sequence
  Synthetic Sequence
accatggacgtactgtttcccctc

SEQ ID NO: 56 - DNA, 24 bp, Artificial Sequence
  Synthetic Sequence
accatggacggtctgtttcccctc

SEQ ID NO: 57 - DNA, 24 bp, Artificial Sequence
  Synthetic Sequence
accatggacgttctgtttcccctc

SEQ ID NO: 58 - DNA, 15 bp, Artificial Sequence
  Synthetic Sequence
cacgttgaggggcat

SEQ ID NO: 59 - DNA, 12 bp, Artificial Sequence
  Synthetic Sequence
tcagcgtgcgcc

SEQ ID NO: 60 - DNA, 17 bp, Artificial Sequence
  Synthetic Sequence
atgacgttcctgacgtt

SEQ ID NO: 61 - DNA, 17 bp, Artificial Sequence
  Synthetic Sequence
tctcccagcgggcgcat

SEQ ID NO: 62 - DNA, 20 bp, Artificial Sequence
  Synthetic Sequence
tccatgtcgttcctgtcgtt

SEQ ID NO: 63 - DNA, 20 bp, Artificial Sequence
  Synthetic Sequence
tccatagcgttcctagcgtt

SEQ ID NO: 64 - DNA, 21 bp, Artificial Sequence
  Synthetic Sequence
tcgtcgctgtctccccttctt

SEQ ID NO: 65 - DNA, 19 bp, Artificial Sequence
  Synthetic Sequence
tcctgacgttcctgacgtt

SEQ ID NO: 66 - DNA, 19 bp, Artificial Sequence
  Synthetic Sequence
tcctgtcgttcctgtcgtt

SEQ ID NO: 67 - DNA, 20 bp, Artificial Sequence
  Synthetic Sequence
tccatgtcgtttttgtcgtt

SEQ ID NO: 68 - DNA, 20 bp, Artificial Sequence
  Synthetic Sequence
tcctgtcgttccttgtcgtt

SEQ ID NO: 69 - DNA, 20 bp, Artificial Sequence
  Synthetic Sequence
tccttgtcgttcctgtcgtt

SEQ ID NO: 70 - DNA, 20 bp, Artificial Sequence
  Synthetic Sequence
tcctgtcgttttttgtcgtt

SEQ ID NO: 71 - DNA, 21 bp, Artificial Sequence
  Synthetic Sequence
tcgtcgctgtctgcccttctt

SEQ ID NO: 72 - DNA, 21 bp, Artificial Sequence
  Synthetic Sequence
tcgtcgctgttgtcgtttctt

SEQ ID NO: 73 - DNA, 20 bp, Artificial Sequence
  Synthetic Sequence
tccatgcgtgcgtgcgtttt

SEQ ID NO: 74 - DNA, 20 bp, Artificial Sequence
  Synthetic Sequence
tccatgcgttgcgttgcgtt

SEQ ID NO: 75 - DNA, 20 bp, Artificial Sequence
  Synthetic Sequence
tccacgacgttttcgacgtt

SEQ ID NO: 76 - DNA, 20 bp, Artificial Sequence
  Synthetic Sequence
tcgtcgttgtcgttgtcgtt

SEQ ID NO: 77 - DNA, 24 bp, Artificial Sequence
  Synthetic Sequence
tcgtcgttttgtcgttttgtcgtt

SEQ ID NO: 78 - DNA, 22 bp, Artificial Sequence
  Synthetic Sequence
tcgtcgttgtcgttttgtcgtt

SEQ ID NO: 79 - DNA, 21 bp, Artificial Sequence
  Synthetic Sequence
gcgtgcgttgtcgttgtcgtt

SEQ ID NO: 80 - DNA, 21 bp, Artificial Sequence
  Synthetic Sequence
tgtcgtttgtcgtttgtcgtt

SEQ ID NO: 81 - DNA, 25 bp, Artificial Sequence
  Synthetic Sequence
tgtcgttgtcgttgtcgttgtcgtt

SEQ ID NO: 82 - DNA, 19 bp, Artificial Sequence
  Synthetic Sequence
tgtcgttgtcgttgtcgtt

SEQ ID NO: 83 - DNA, 14 bp, Artificial Sequence
  Synthetic Sequence
tcgtcgtcgtcgtt

SEQ ID NO: 84 - DNA, 13 bp, Artificial Sequence
  Synthetic Sequence
tgtcgttgtcgtt

SEQ ID NO: 85 - DNA, 20 bp, Artificial Sequence
  Synthetic Sequence
tccatagcgttcctagcgtt

SEQ ID NO: 86 - DNA, 20 bp, Artificial Sequence
  Synthetic Sequence
tccatgacgttcctgacgtt

SEQ ID NO: 87 - DNA, 6 bp, Artificial Sequence
  Synthetic Sequence
gtcgyt

SEQ ID NO: 88 - DNA, 7 bp, Artificial Sequence
  Synthetic Sequence
tgtcgyt

SEQ ID NO: 89 - DNA, 18 bp, Artificial Sequence
  Synthetic Sequence
agctatgacgttccaagg

SEQ ID NO: 90 - DNA, 20 bp, Artificial Sequence
  Synthetic Sequence
tccatgacgttcctgacgtt

SEQ ID NO: 91 - DNA, 20 bp, Artificial Sequence
  Synthetic Sequence
atcgactctcgaacgttctc

SEQ ID NO: 92 - DNA, 20 bp, Artificial Sequence
  Synthetic Sequence
tccatgtcggtcctgacgca

SEQ ID NO: 93 - DNA, 8 bp, Artificial Sequence
  Synthetic Sequence
tcttcgat

SEQ ID NO: 94 - DNA, 20 bp, Artificial Sequence
  Synthetic Sequence
ataggaggtccaacgttctc

SEQ ID NO: 95 - DNA, 15 bp, Artificial Sequence
  Synthetic Sequence
gctagaggggagggt

SEQ ID NO: 96 - DNA, 15 bp, Artificial Sequence
  Synthetic Sequence
gctagatgttagggg

SEQ ID NO: 97 - DNA, 15 bp, Artificial Sequence
  Synthetic Sequence
gctagaggggagggt

SEQ ID NO: 98 - DNA, 15 bp, Artificial Sequence
  Synthetic Sequence
gctagaggggagggt

SEQ ID NO: 99 - DNA, 15 bp, Artificial Sequence
  Synthetic Sequence
gcatgagggggagct

SEQ ID NO: 100 - DNA, 20 bp, Artificial Sequence
  Synthetic Sequence
atggaaggtccagggggctc

SEQ ID NO: 101 - DNA, 20 bp, Artificial Sequence
  Synthetic Sequence
atggactctggagggggctc

SEQ ID NO: 102 - DNA, 20 bp, Artificial Sequence
  Synthetic Sequence
atggactctggagggggctc

SEQ ID NO: 103 - DNA, 20 bp, Artificial Sequence
  Synthetic Sequence
atggactctggagggggctc

SEQ ID NO: 104 - DNA, 20 bp, Artificial Sequence
  Synthetic Sequence
atggaaggtccaaggggctc

SEQ ID NO: 105 - DNA, 20 bp, Artificial Sequence
  Synthetic Sequence
gagaaggggggaccttccat

SEQ ID NO: 106 - DNA, 20 bp, Artificial Sequence
  Synthetic Sequence
gagaaggggggaccttccat

SEQ ID NO: 107 - DNA, 20 bp, Artificial Sequence
  Synthetic Sequence
gagaaggggggaccttggat

SEQ ID NO: 108 - DNA, 20 bp, Artificial Sequence
  Synthetic Sequence
gagaaggggggaccttccat

SEQ ID NO: 109 - DNA, 20 bp, Artificial Sequence
  Synthetic Sequence
gagaaggggggaccttccat

SEQ ID NO: 110 - DNA, 20 bp, Artificial Sequence
  Synthetic Sequence
gagaaggggccagcactgat

SEQ ID NO: 111 - DNA, 20 bp, Artificial Sequence
  Synthetic Sequence
tccatgtggggcctgatgct

SEQ ID NO: 112 - DNA, 20 bp, Artificial Sequence
  Synthetic Sequence
tccatgtggggcctgatgct

SEQ ID NO: 113 - DNA, 20 bp, Artificial Sequence
  Synthetic Sequence
tccatgaggggcctgatgct

SEQ ID NO: 114 - DNA, 20 bp, Artificial Sequence
  Synthetic Sequence
tccatgtggggcctgctgat

SEQ ID NO: 115 - DNA, 20 bp, Artificial Sequence
  Synthetic Sequence
atggactctccggggttctc

SEQ ID NO: 116 - DNA, 20 bp, Artificial Sequence
  Synthetic Sequence
atggaaggtccggggttctc

SEQ ID NO: 117 - DNA, 20 bp, Artificial Sequence
  Synthetic Sequence
atggactctggaggggtctc

SEQ ID NO: 118 - DNA, 20 bp, Artificial Sequence
  Synthetic Sequence
atggaggctccatggggctc

SEQ ID NO: 119 - DNA, 20 bp, Artificial Sequence
  Synthetic Sequence
atggactctggggggttctc

SEQ ID NO: 120 - DNA, 20 bp, Artificial Sequence
  Synthetic Sequence
atggactctggggggttctc

SEQ ID NO: 121 - DNA, 20 bp, Artificial Sequence
  Synthetic Sequence
tccatgtgggtggggatgct

SEQ ID NO: 122 - DNA, 20 bp, Artificial Sequence
  Synthetic Sequence
tccatgcgggtggggatgct

SEQ ID NO: 123 - DNA, 20 bp, Artificial Sequence
  Synthetic Sequence
tccatgggggtcctgatgct

SEQ ID NO: 124 - DNA, 20 bp, Artificial Sequence
  Synthetic Sequence
tccatgggggtcctgatgct

SEQ ID NO: 125 - DNA, 20 bp, Artificial Sequence
  Synthetic Sequence
tccatgtggggcctgatgct

SEQ ID NO: 126 - DNA, 20 bp, Artificial Sequence
  Synthetic Sequence
tccatgtggggcctgatgct

SEQ ID NO: 127 - DNA, 20 bp, Artificial Sequence
  Synthetic Sequence
tccatggggtccctgatgct

SEQ ID NO: 128 - DNA, 20 bp, Artificial Sequence
  Synthetic Sequence
tccatggggtgcctgatgct

SEQ ID NO: 129 - DNA, 20 bp, Artificial Sequence
  Synthetic Sequence
tccatggggttcctgatgct

SEQ ID NO: 130 - DNA, 20 bp, Artificial Sequence
  Synthetic Sequence
tccatggggtccctgatgct

SEQ ID NO: 131 - DNA, 20 bp, Artificial Sequence
  Synthetic Sequence
tccatcgggggcctgatgct

SEQ ID NO: 132 - DNA, 14 bp, Artificial Sequence
  Synthetic Sequence
gctagagggagtgt

SEQ ID NO: 133 - DNA, 20 bp, Artificial Sequence
  Synthetic Sequence
gggggggggggggggggggg

SEQ ID NO: 134 - DNA, 21 bp, Artificial Sequence
  Synthetic Sequence
actgacagactgacagactga

SEQ ID NO: 135 - DNA, 21 bp, Artificial Sequence
  Synthetic Sequence
agtgacagacagacacactga

SEQ ID NO: 136 - DNA, 21 bp, Artificial Sequence
  Synthetic Sequence
actgacagactgatagaccca

SEQ ID NO: 137 - DNA, 21 bp, Artificial Sequence
  Synthetic Sequence
agtgagagactgcaagactga

SEQ ID NO: 138 - DNA, 21 bp, Artificial Sequence
  Synthetic Sequence
aatgccagtccgacaggctga

SEQ ID NO: 139 - DNA, 21 bp, Artificial Sequence
  Synthetic Sequence
ccagaacagaagcaatggatg

SEQ ID NO: 140 - DNA, 21 bp, Artificial Sequence
  Synthetic Sequence
cctgaacagaagccatggatg

SEQ ID NO: 141 - DNA, 21 bp, Artificial Sequence
  Synthetic Sequence
gcagaacagaagacatggatg

SEQ ID NO: 142 - DNA, 21 bp, Artificial Sequence
  Synthetic Sequence
ccacaacacaagcaatggata

SEQ ID NO: 143 - DNA, 21 bp, Artificial Sequence
  Synthetic Sequence
aagctagccagctagctagca

SEQ ID NO: 144 - DNA, 21 bp, Artificial Sequence
  Synthetic Sequence
cagctagccacctagctagca

SEQ ID NO: 145 - DNA, 21 bp, Artificial Sequence
  Synthetic Sequence
aagctaggcagctaactagca

SEQ ID NO: 146 - DNA, 21 bp, Artificial Sequence
  Synthetic Sequence
gagctagcaagctagctagga

(19) United States
(12) Patent Application Publication
(10) Pub. No.: US 2006/0211639 A1
(43) Pub. Date: Sep. 21, 2006

(54) Immunostimulatory nucleic acids and cancer medicament combination therapy for the treatment of cancer

(76) Inventors: Robert L. Bratzler, Concord, MA (US); Deanna M. Petersen, Newton, MA (US)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 10/668,050

(22) Filed: Sep. 22, 2003

Related U.S. Application Data
(63) Continuation of application No. 09/800,266, filed on Mar. 5, 2001, now abandoned.
(60) Provisional application No. 60/187,214, filed on Mar. 3, 2000.

(51) Int. Cl. A61K 48/00; A61K 38/16; A61K 31/7072; A61K 31/704; A61K 31/4745; A61K 31/337; A61K 31/282

(52) U.S. Cl. 514/44; 514/34; 514/449; 424/649; 514/50; 514/283; 514/492; 514/8; 514/410; 514/589

(57) ABSTRACT

The invention involves administration of an immunostimulatory nucleic acid in combination with a cancer medicament for the treatment or prevention of cancer in subjects. The combination of drugs are administered in synergistic amounts or in various dosages or at various time schedules. The invention also relates to kits and compositions concerning the combination of drugs.

### RELATED APPLICATIONS

This application claims priority to and is a continuation of co-pending U.S. Ser. No. 09/800,266 filed on Mar. 5, 2001, which claims priority under Title 35 §119(e) of the U.S. Provisional Application No. 60/187,214, filed Mar. 3, 2000, and entitled “Immunostimulatory Nucleic Acids and Cancer Medicament Combination Therapy for the Treatment of Cancer”, the entire contents of which are incorporated herein by reference.

### FIELD OF THE INVENTION

The present invention relates to the use of immunostimulatory nucleic acids in combination with cancer medicaments in the treatment of cancer.

### BACKGROUND OF THE INVENTION

Cancer is the second leading cause of death, resulting in one out of every four deaths, in the United States. In 1997, the estimated total number of new diagnoses for lung, breast, prostate, colorectal and ovarian cancer was approximately two million. Due to the ever increasing aging population in the United States, it is reasonable to expect that rates of cancer incidence will continue to grow.

Cancer is a disease which involves the uncontrolled growth (i.e., division) of cells. Some of the known mechanisms which contribute to the uncontrolled proliferation of cancer cells include growth factor independence, failure to detect genomic mutation, and inappropriate cell signaling. The ability of cancer cells to ignore normal growth controls may result in an increased rate of proliferation. Although the causes of cancer have not been firmly established, there are some factors known to contribute, or at least predispose a subject, to cancer. Such factors include particular genetic mutations (e.g., BRCA gene mutation for breast cancer, APC for colon cancer), exposure to suspected cancer-causing agents, or carcinogens (e.g., asbestos, UV radiation) and familial disposition for particular cancers such as breast cancer.

Cancer is currently treated using a variety of modalities including surgery, radiation therapy and chemotherapy. The choice of treatment modality will depend upon the type, location and dissemination of the cancer. For example, surgery and radiation therapy may be of non-solid tumor cancers such as leukemia and lymphoma. One of the advantages of surgery and radiation therapy is the ability to control to some extent the impact of the therapy, and thus to limit the toxicity to normal tissues in the body. However, surgery and radiation therapy are often followed by chemotherapy to guard against any remaining or radio-resistant cancer cells. Chemotherapy is also the most appropriate treatment for disseminated cancers such as leukemia and lymphoma as well as metastases.

Chemotherapy refers to therapy using chemical and/or biological agents to attack cancer cells. Unlike localized surgery or radiation, chemotherapy is generally administered in a systemic fashion and thus toxicity to normal tissues is a major concern. Because many chemotherapy agents target cancer cells based on their proliferative profiles, tissues such as the gastrointestinal tract and the bone marrow which are normally proliferative are also susceptible to the effects of the chemotherapy. One of the major side effects of chemotherapy is myelosuppression (including anemia, neutropenia and thrombocytopenia) which results from the death of normal hemopoietic precursors.

Many chemotherapeutic agents have been developed for the treatment of cancer. Not all tumors, however, respond to chemotherapeutic agents and others although initially responsive to chemotherapeutic agents may develop resistance. As a result, the search for effective anti-cancer drugs has intensified in an effort to find even more effective agents with less non-specific toxicity.

Recently, it has been shown that nucleic acid molecules having a CpG dinucleotide motif in which the C is unmethylated are also useful in the prevention and treatment of cancer (U.S. Pat. No. 6,194,388). These nucleic acid molecules are believed to stimulate innate immune responses against cancer cells, as well as acting as adjuvants for the induction of specific immune responses to cancer cells.

### SUMMARY OF THE INVENTION

The invention provides improved methods and products for the treatment of subjects having cancer or at risk of developing cancer. The invention is based, in part, on the finding that when some types of immunostimulatory nucleic acid molecules are used in conjunction with some forms of cancer medicament, some unexpected and improved results are observed. For instance, the efficacy of the combination of some immunostimulatory nucleic acids and some cancer medicaments is profoundly improved over the use of the cancer medicament alone. The results are surprising, in part, because the immunostimulatory nucleic acids and the cancer medicaments act through different mechanisms and would not necessarily be expected to improve the efficacy of the other in a synergistic manner.

In one aspect, the invention provides a method for treating a subject having, or at risk of developing, a cancer, comprising administering to a subject in need of such treatment a poly-G nucleic acid and a cancer medicament in an effective amount to treat the cancer or to reduce the risk of developing the cancer. The poly-G nucleic acid is not conjugated to the cancer medicament.

In certain embodiments of some aspects of the invention, unless otherwise indicated, the cancer medicament embraces at least one or more chemotherapeutic agents, immunotherapeutic agents, cancer vaccines, biological response modifiers (e.g., cytokines and hemopoietic growth factors), or hormone therapies (e.g., adrenocorticosteroids, androgens, anti-androgens, estrogens, anti-estrogens, progestins, aromatase inhibitor, gonadotropin-releasing hormone agonists, and somatostatin analogs).

In one embodiment, the cancer medicament is a chemotherapeutic agent selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Placlitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate. In an important embodiment, the cancer medicament is taxol.

In another embodiment, the cancer medicament is an immunotherapeutic agent selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA.

In yet another embodiment, the cancer medicament is a cancer vaccine selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys.

In still another embodiment, the cancer medicament is a hormone therapy. In a related embodiment, the hormone therapy is selected from the group consisting of estrogen therapy e.g., diethylstilbestrol and ethinyl estradiol, anti-estrogen therapy e.g., tamoxifen, progestin therapy e.g., medroxyprogesterone and megestrol acetate, androgen blockade e.g., anti-androgens such as flutamide, adrenocorticosteroids including adrenal steroids, synthetic glucocorticoid therapy e.g., prednisone, methylprednisone, and dexamethasone, androgens e.g., fluoxymesterone, synthetic testosterone analogs, aromatase inhibitor e.g., aminoglutethimide, gonadotropin-releasing hormone agonists e.g., leuprolide, somatostatin analogs e.g., octreotide. In certain embodiments, the method further comprises administering interferon-α to the subject. The cancer may be selected from the group consisting of bone cancer, brain and CNS cancer, connective tissue cancer, esophageal cancer, eye cancer, Hodgkin's lymphoma, larynx cancer, oral cavity cancer, skin cancer, and testicular cancer, but it is not so limited.

In certain embodiments, the immunostimulatory nucleic acid has a modified backbone. The modified backbone may be a phosphorothioate modified backbone.

In another aspect, the invention provides another method for treating a subject having or at risk of developing a cancer. This method comprises administering to a subject in need of such treatment, an immunostimulatory nucleic acid having a modified backbone and a cancer medicament selected from the group consisting of an immunotherapeutic agent, a cancer vaccine and a hormone therapy. The immunostimulatory nucleic acid is free of a CpG motif, and a T-rich motif. In one embodiment, the cancer medicament is taxol.

In certain embodiments, the method further comprises administering interferon-α to the subject. In other embodiments, the method further comprises administering a cancer antigen to the subject. In some embodiments, the cancer antigen is not conjugated to the immunostimulatory nucleic acid.

In one embodiment, the immunostimulatory nucleic acid is a poly-G nucleic acid. In a related embodiment, the poly-G nucleic acid is not conjugated to the cancer medicament. In another embodiment, the immunostimulatory nucleic acid has a nucleotide sequence selected from the group consisting of SEQ ID NO:134 through to SEQ. ID NO:146. The immunostimulatory nucleic acid may have a modified backbone such as, but not limited to, a phosphorothioate modified backbone.

In yet a further aspect, the invention provides yet another method for treating a subject having or at risk of developing cancer. This method comprises administering to a subject in need of such treatment an immunostimulatory nucleic acid selected from the group consisting of a CpG nucleic acid and a non-CpG nucleic acid, and a hormone therapy. The hormone therapy may be selected from the group consisting of estrogen therapy e.g., diethylstilbestrol and ethinyl estradiol, anti-estrogen therapy e.g., tamoxifen, progestin therapy e.g., medroxyprogesterone and megestrol acetate, androgen blockade e.g., anti-androgens such as flutamide, adrenocorticosteroids including adrenal steroids, synthetic glucocorticoid therapy e.g., prednisone, methylprednisone, and dexamethasone, androgens e.g., fluoxymesterone, synthetic testosterone analogs, aromatase inhibitor e.g., aminoglutethimide, gonadotropin-releasing hormone agonists e.g., leuprolide, somatostatin analogs e.g., octreotide. As used herein, a non-CpG nucleic acid is an immunostimulatory nucleic acid that does not possess a methylated or an unmethylated CpG motif, and preferably also does not possess a T-rich motif and/or a poly-G motif. In important embodiments, a non-CpG nucleic acid is a nucleic acid capable of stimulating a Th2 immune response.

In one embodiment, the method further comprising administering a cancer antigen to the subject. In certain embodiments, the cancer antigen is not conjugated to the immunostimulatory nucleic acid.

In one embodiment, the immunostimulatory nucleic acid has a modified backbone. The modified backbone may be a phosphorothioate modified backbone, but it is not so limited.

In yet another aspect, the invention provides a method for preventing an allergic reaction in a subject receiving a blood transfusion, comprising administering to a subject receiving a blood transfusion an immunostimulatory nucleic acid in an effective amount to prevent an allergic reaction to the blood transfusion.

In one embodiment, the blood transfusion is a red blood cell transfusion. In another embodiment, the blood transfusion is a platelet transfusion.

In one embodiment, the immunostimulatory nucleic acid is a CpG nucleic acid. In another embodiment, the immunostimulatory nucleic acid has a modified backbone. The modified backbone may be a phosphorothioate modified backbone, but it is not so limited. In a related embodiment, the immunostimulatory nucleic acid with the phosphorothioate modified backbone is free of a CpG motif, and a T-rich motif. In still another embodiment, the immunostimulatory nucleic acid is not a poly-G nucleic acid.

In one embodiment, the subject has cancer. In another embodiment, the subject is anemic or thrombocytopenic.

In a related aspect, the invention provides a device for delivering an immunostimulatory nucleic acid to a subject receiving an intravenous injection, comprising an intravenous device selected from the group consisting of an intravenous bag and an intravenous tube, and an immunostimulatory nucleic acid. The immunostimulatory nucleic acid is coated on an internal surface of the intravenous device or is embedded within the intravenous device. In this latter configuration, the intravenous bag or tubing acts as a sustained release device for the sustained delivery of the immunostimulatory nucleic acid.

In some aspects of the invention, the immunostimulatory nucleic acids and the cancer medicaments are administered as a synergistic combination in an effective amount to treat or reduce the risk of developing a cancer. As used herein, the term “synergistic” describes an effect resulting from the combination of at least two agents which is greater than the effect of each of the individual agents when used alone. It was surprisingly discovered according to the invention that select combinations of immunostimulatory nucleic acids and the cancer medicaments worked synergistically to treat and reduce the risk of developing a cancer.

In certain embodiments of all aspects of the invention, the immunostimulatory nucleic acid may be a nucleic acid which stimulates a Th1 immune response. Similarly, in some aspects of the invention, it is conceivable that one or more cancer medicaments can be administered to a subject. Thus depending on the embodiment, one, two, three, four, five or more cancer medicaments may be administered to a subject in a particular method. Thus, the term “a cancer medicament” is meant to embrace a single medicament, a plurality of medicaments of a particular class and a plurality of medicaments of different classes.

According to other embodiments, the immunostimulatory nucleic acid is administered concurrently with, prior to, or following the administration of the cancer medicament.

In some embodiments, the immunostimulatory nucleic acid is administered in an effective amount for upregulating, enhancing or activating an immune response. In some embodiments, the immunostimulatory nucleic acid is administered in an effective amount for redirecting the immune response from a Th2 to a Th1 immune response. In other embodiments, the immunostimulatory nucleic acid is administered in an effective amount for redirecting the immune response from a Th1 to a Th2 immune response. In still other embodiments, a plurality of immunostimulatory nucleic acids, with different nucleic acid sequences and with different functional effects, is administered.

Each of the limitations of the invention can encompass various embodiments of the invention. It is, therefore, anticipated that each of the limitations of the invention involving any one element or combinations of elements can be included in each aspect of the invention.

### DETAILED DESCRIPTION OF THE INVENTION

Present cancer treatments are too often ineffective as well as being associated with a high degree of patient morbidity, most probably due to a lack of toxic specificity for tumor cells. The invention provides methods and products for the more effective treatment of cancer using some immunostimulatory nucleic acids in combination with some cancer medicaments. In some instances, the combination of the immunostimulatory nucleic acid and cancer medicament is synergistic, resulting in greater than additive effects than would otherwise be expected using the agents separately.

The invention is based, in part, on the surprising discovery that administration of some immunostimulatory nucleic acids with some cancer medicaments to a subject having cancer or at risk of developing cancer has synergistic anti-cancer activity. Thus, in one aspect, the invention provides a method for treating or preventing cancer which involves the administration of some forms of immunostimulatory nucleic acid and some forms of cancer medicament in an effective amount to prevent or treat the cancer to a subject having cancer or a subject at risk of developing cancer.

In one aspect of the invention, the combination of immunostimulatory nucleic acids and cancer medicaments allows for the administration of higher doses of cancer medicaments without as many side effects as are ordinarily experienced at those high doses. In another aspect, the combination of immunostimulatory nucleic acids and cancer medicaments allows for the administration of lower, sub-therapeutic doses of either compound, but with higher efficacy than would otherwise be achieved using such low doses. As one example, by administering a combination of an immunostimulatory nucleic acid and a cancer medicament, it is possible to achieve an effective anti-cancer response even though the cancer medicament is administered at a dose which alone would not provide a therapeutic benefit (i.e., a sub-therapeutic dose). As another example, the combined administration achieves an anti-cancer response even though the immunostimulatory nucleic acid is administered at a dose which alone would not provide a therapeutic benefit.

An “immunostimulatory nucleic acid” as used herein is any nucleic acid containing an immunostimulatory motif or backbone that induces an immune response. The immune response may be characterized as, but is not limited to, a Th1-type immune response or a Th2-type immune response. Such immune responses are defined by cytokine and antibody production profiles which are elicited by the activated immune cells.

Helper (CD4⁺) T cells orchestrate the immune response of mammals through production of soluble factors that act on other immune system cells, including other T cells. Helper CD4⁺, and in some instances also CD8⁺, T cells are characterized as Th1 and Th2 cells in both murine and human systems, depending on their cytokine production profiles (Romagnani, 1991, Immunol Today 12: 256-257, Mosmann, 1989, Annu Rev Immunol, 7: 145-173). Th1 cells produce interleukin 2 (IL-2), IL-12, tumor necrosis factor (TNFα) and interferon gamma (IFN-γ) and they are responsible primarily for cell-mediated immunity such as delayed type hypersensitivity. The cytokines that are induced by administration of immunostimulatory nucleic acids are predominantly of the Th1 class. The types of antibodies associated with a Th1 response are generally more protective because they have high neutralization and opsonization capabilities. Th2 cells produce IL-4, IL-5, IL-6, IL-9, IL-10 and IL-13 and are primarily involved in providing optimal help for humoral immune responses such as IgE and IgG4 antibody isotype switching (Mosmann, 1989, Annu Rev Immunol, 7: 145-173). Th2 responses involve predominantly antibodies that have less protective effects against infection.

The terms “nucleic acid” and “oligonucleotide” are used interchangeably to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acids include vectors, e.g., plasmids, as well as oligonucleotides. Nucleic acid molecules can be obtained from existing nucleic acid sources (e.g., genomic or cDNA, referred to as isolated nucleic acids), but are preferably synthetic (e.g. produced by oligonucleotide synthesis).

Immunostimulatory nucleic acids may possess immunostimulatory motifs such as CpG motif, and poly-G motifs. In some embodiments of the invention, any nucleic acid, regardless of whether it possesses an identifiable motif, can be used in the combination therapy to elicit an immune response. Immunostimulatory backbones include, but are not limited to, phosphate modified backbones, such as phosphorothioate backbones. Immunostimulatory nucleic acids have been described extensively in the prior art and a brief summary of these nucleic acids is presented below. Most aspects of the invention, particularly those directed at treating subjects having or at risk of developing cancer, do not embrace the use of T-rich or methylated CpG nucleic acids (i.e., nucleic acids that possess either a T-rich or a methylated CpG motif).

In some embodiments, a CpG immunostimulatory nucleic acid is used in the methods of the invention. A CpG immunostimulatory nucleic acid is a nucleic acid which contains a CG dinucleotide, the C residue of which is unmethylated. CpG immunostimulatory nucleic acids are known to stimulate Th1-type immune responses. CpG sequences, while relatively rare in human DNA are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG containing nucleic acids, relying on this innate immune defense mechanism can utilize a unique and natural pathway for immune therapy. The effects of CpG nucleic acids on immune modulation have been described extensively in U.S. Pat. No. 6,194,388, and published patent applications, such as PCT US95/01570, PCT/US97/19791, PCT/US98/03678, PCT/US98/10408, PCT/US98/04703, PCT/US99/07335, and PCT/US99/09863. The entire contents of each of these issued patents and patent applications are hereby incorporated by reference.

A CpG nucleic acid is a nucleic acid which includes at least one unmethylated CpG dinucleotide. A nucleic acid containing at least one unmethylated CpG dinucleotide is a nucleic acid molecule which contains an unmethylated cytosine in a cytosine-guanine dinucleotide sequence (i.e. “CpG DNA” or DNA containing a 5′ cytosine followed by 3′ guanosine and linked by a phosphate bond) and activates the immune system. The CpG nucleic acids can be double-stranded or single-stranded. Generally, double-stranded molecules are more stable in vivo, while single-stranded molecules have increased immune activity. Thus in some aspects of the invention it is preferred that the nucleic acid be single stranded and in other aspects it is preferred that the nucleic acid be double stranded. The terms CpG nucleic acid or CpG oligonucleotide as used herein refer to an immunostimulatory CpG nucleic acid unless otherwise indicated. The entire immunostimulatory nucleic acid can be unmethylated or portions may be unmethylated but at least the C of the 5′ CG 3′ must be unmethylated.

In one preferred embodiment the invention provides an immunostimulatory nucleic acid which is a CpG nucleic acid represented by at least the formula:
5′X₁X₂CGX₃X₄3′
wherein X₁, X₂, X₃, and X₄ are nucleotides. In one embodiment X₂ is adenine, guanine, cytosine, or thymine. In another embodiment X₃ is cytosine, guanine, adenine, or thymine. In other embodiments X₂ is adenine, guanine, or thymine and X₃ is cytosine, adenine, or thymine.

In another embodiment the immunostimulatory nucleic acid is an isolated CpG nucleic acid represented by at least the formula:
5′N₁X₁X₂CGX₃X₄N₂3′
wherein X₁, X₂, X₃, and X₄ are nucleotides and N is any nucleotide and N₁ and N₂ are nucleic acid sequences composed of from about 0-25 N's each. In one embodiment X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, ApT, TpG, ApG, CpG, TpC, ApC, CpC, TpA, ApA, and CpA. Preferably X₁X₂ are GpA or GpT and X₃X₄ are TpT. In other embodiments X₁ or X₂ or both are purines and X₃ or X₄ or both are pyrimidines or X₁X₂ are GpA and X₃ or X₄ or both are pyrimidines. In another preferred embodiment X₁X₂ are nucleotides selected from the group consisting of: TpA, ApA, ApC, ApG, and GpG. In yet another embodiment X₃X₄ are nucleotides selected from the group consisting of: TpT, TpA, TpG, ApA, ApG, ApC, and CpA. X₁X₂ in another embodiment are nucleotides selected from the group consisting of: TpT, TpG, ApT, GpC, CpC, CpT, TpC, GpT and CpG.

In another preferred embodiment the immunostimulatory nucleic acid has the sequence 5′TCN₁TX₁X₂CGX₃X₄3′. The immunostimulatory nucleic acids of the invention in some embodiments include X₁X₂ selected from the group consisting of GpT, GpG, GpA and ApA and X₃X₄ is selected from the group consisting of TpT, CpT and TpC.

For facilitating uptake into cells, the immunostimulatory nucleic acids are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

“Palindromic sequence” shall mean an inverted repeat (i.e., a sequence such as ABCDEE′D′C′B′A′ in which A and A′ are bases capable of forming the usual Watson-Crick base pairs). In vivo, such sequences may form double-stranded structures. In one embodiment the CpG nucleic acid contains a palindromic sequence. A palindromic sequence used in this context refers to a palindrome in which the CpG is part of the palindrome, and preferably is the center of the palindrome. In another embodiment the CpG nucleic acid is free of a palindrome. An immunostimulatory nucleic acid that is free of a palindrome is one in which the CpG dinucleotide is not part of a palindrome. Such an oligonucleotide may include a palindrome in which the CpG is not the center of the palindrome.

The CpG nucleic acid sequences of the invention are those broadly described above as well as disclosed in PCT Published Patent Applications PCT/US95/01570 and PCT/US97/19791 claiming priority to U.S. Ser. Nos. 08/386,063 and 08/960,774, filed on Feb. 7, 1995 and October 30, 1997 respectively.

In some embodiments of the invention, a non-CpG immunostimulatory nucleic acid is used. A non-CpG immunostimulatory nucleic acid is a nucleic acid which does not have a CpG motif in its sequence, regardless of whether the C is the dinucleotide is methylated or unmethylated. Non-CpG immunostimulatory nucleic acids may induce Th1 or Th2 immune responses, depending upon their sequence, their mode of delivery and the dose at which they are administered.

An important subset of non-CpG immunostimulatory nucleic acids are poly-G immunostimulatory nucleic acids. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J. Biochem. 116, 991-994 also describe the immunostimulatory properties of poly-G nucleic acids. In accordance with one aspect of the invention, poly-G-containing nucleotides are useful, inter alia, for treating and preventing bacterial, viral and fungal infections, and can thereby be used to minimize the impact of these infections on the treatment of cancer patients.

Poly-G nucleic acids preferably are nucleic acids having the following formulas:
5′X₁X₂GGGX₃X₄3′
wherein X₁, X₂, X₃, and X₄ are nucleotides. In preferred embodiments at least one of X₃ and X₄ are a G. In other embodiments both of X₃ and X₄ are a G. In yet other embodiments the preferred formula is 5′GGGNGGG3′, or 5′GGGNGGGNGGG3′ wherein N represents between 0 and 20 nucleotides. In other embodiments the Poly-G nucleic acid is free of unmethylated CG dinucleotides, such as, for example, the nucleic acids listed above as SEQ ID NO: 95 through to SEQ ID NO: 133. In other embodiments the Poly-G nucleic acid includes at least one unmethylated CG dinucleotide, such as, for example, the nucleic acids listed below as SEQ ID NO: 46, SEQ ID NO: 47, SEQ ID NO: 58, and SEQ ID NO: 61.

The immunostimulatory nucleic acids of the invention can also be those which do not possess CpG, poly-G, or T-rich motifs. Examples of such nucleic acid sequences are listed below as SEQ ID NO: 134 through to SEQ ID NO: 146. T-rich motifs and nucleic acids possessing such motifs are described in U.S. patent application Ser. No. 09/669,187 filed Sep. 25, 2000, by Krieg et al., the entire contents of which are incorporated herein by reference. Other non-CpG nucleic acids are described in U.S. patent application Ser. No. 09/768,012, filed Jan. 22, 2001, the entire contents of which are incorporated herein in their entirety.

Exemplary immunostimulatory nucleic acid sequences include but are not limited to those immunostimulatory sequences shown in Table 1.

TABLE 1

| | |
|---|---|
| GCTAGACGTTAGCGT; | (SEQ ID NO: 1) |
| GCTAGATGTTAGCGT; | (SEQ ID NO: 2) |
| GCTAGACGTTAGCGT; | (SEQ ID NO: 3) |
| GCTAGACGTTAGCGT; | (SEQ ID NO: 4) |
| GCATGACGTTGAGCT; | (SEQ ID NO: 5) |
| ATGGAAGGTCCAGCGTTCTC; | (SEQ ID NO: 6) |
| ATCGACTCTCGAGCGTTCTC; | (SEQ ID NO: 7) |
| ATCGACTCTCGAGCGTTCTC; | (SEQ ID NO: 8) |
| ATCGACTCTCGAGCGTTCTC; | (SEQ ID NO: 9) |
| ATGGAAGGTCCAACGTTCTC; | (SEQ ID NO: 10) |
| GAGAACGCTGGACCTTCCAT; | (SEQ ID NO: 11) |
| GAGAACGCTCGACCTTCCAT; | (SEQ ID NO: 12) |
| GAGAACGCTCGACCTTCGAT; | (SEQ ID NO: 13) |
| GAGAACGCTGGACCTTCCAT; | (SEQ ID NO: 14) |
| GAGAACGATGGACCTTCCAT; | (SEQ ID NO: 15) |
| GAGAACGCTCCAGCACTGAT; | (SEQ ID NO: 16) |
| TCCATGTCGGTCCTGATGCT; | (SEQ ID NO: 17) |
| TCCATGTCGGTCCTGATGCT; | (SEQ ID NO: 18) |
| TCCATGACGTTCCTGATGCT; | (SEQ ID NO: 19) |
| TCCATGTCGGTCCTGCTGAT; | (SEQ ID NO: 20) |
| TCAACGTT; | (SEQ ID NO: 21) |
| TCAGCGCT; | (SEQ ID NO: 22) |
| TCATCGAT; | (SEQ ID NO: 23) |
| TCTTCGAA; | (SEQ ID NO: 24) |
| CAACGTT; | (SEQ ID NO: 25) |
| CCAACGTT; | (SEQ ID NO: 26) |
| AACGTTCT; | (SEQ ID NO: 27) |
| TCAACGTC; | (SEQ ID NO: 28) |
| ATGGACTCTGCAGCGTTGTC; | (SEQ ID NO: 29) |
| ATGGAAGGTCCAACGTTCTC; | (SEQ ID NO: 30) |
| ATCGACTCTCGAGCGTTCTG; | (SEQ ID NO: 31) |
| ATGGAGGCTCCATCGTTCTC; | (SEQ ID NO: 32) |
| ATCGACTCTCGAGCGTTCTC; | (SEQ ID NO: 33) |
| ATCGACTCTCGAGCGTTCTC; | (SEQ ID NO: 34) |
| TCCATGTCGGTCCTGATGCT; | (SEQ ID NO: 35) |
| TCCATGCCGGTCCTGATGCT; | (SEQ ID NO: 36) |
| TCCATGGCGGTCCTGATGCT; | (SEQ ID NO: 37) |
| TCCATGACGGTCCTGATGCT; | (SEQ ID NO: 38) |
| TCCATGTCGATCCTGATGCT; | (SEQ ID NO: 39) |
| TCCATGTGGCTCCTGATGCT; | (SEQ ID NO: 40) |
| TCCATGTCGTCCCTGATGCT; | (SEQ ID NO: 41) |
| TCCATGACGTGCCTGATGCT; | (SEQ ID NO: 42) |
| TCCATAACGTTCCTGATGCT; | (SEQ ID NO: 43) |
| TCCATGACGTCCCTGATGCT; | (SEQ ID NO: 44) |
| TCCATCACGTGCCTGATGCT; | (SEQ ID NO: 45) |
| GGGGTCAACGTTGACGGGG; | (SEQ ID NO: 46) |
| GGGGTCAGTCGTGACGGGG; | (SEQ ID NO: 47) |
| GCTAGACGTTAGTGT; | (SEQ ID NO: 48) |
| TCCATGTCGTTCCTGATGCT; | (SEQ ID NO: 49) |
| ACCATGGACGATCTGTTTCCCCTC; | (SEQ ID NO: 50) |
| TCTCCCAGCGTGCGCCAT; | (SEQ ID NO: 51) |
| ACCATGGACGAACTGTTTCCCCTC; | (SEQ ID NO: 52) |
| ACCATGGACGAGCTGTTTCCCCTC; | (SEQ ID NO: 53) |
| ACCATGGACGACCTGTTTCCCCTC; | (SEQ ID NO: 54) |
| ACCATGGACGTACTGTTTCCCCTC; | (SEQ ID NO: 55) |
| ACCATGGACGGTCTGTTTCCCCTC; | (SEQ ID NO: 56) |
| ACCATGGACGTTCTGTTTCCCCTC; | (SEQ ID NO: 57) |
| CACGTTGAGGGGCAT; | (SEQ ID NO: 58) |
| TCAGCGTGCGCC; | (SEQ ID NO: 59) |
| ATGACGTTCCTGACGTT; | (SEQ ID NO: 60) |
| TCTCCCAGCGGGCGCAT; | (SEQ ID NO: 61) |
| TCCATGTCGTTCCTGTCGTT; | (SEQ ID NO: 62) |
| TCCATAGCGTTCCTAGCGTT; | (SEQ ID NO: 63) |
| TCGTCGCTGTCTCCCCTCTT; | (SEQ ID NO: 64) |
| TCCTGACGTTCCTGACGTT; | (SEQ ID NO: 65) |
| TCCTGTCGTTCCTGTCGTT; | (SEQ ID NO: 66) |
| TCCATGTCGTTTTTGTCGTT; | (SEQ ID NO: 67) |
| TCCTGTCGTTCCTTGTCGTT; | (SEQ ID NO: 68) |
| TCCTTGTCGTTCCTGTCGTT; | (SEQ ID NO: 69) |
| TCCTGTCGTTTTTTGTCGTT; | (SEQ ID NO: 70) |
| TCGTCGCTGTCTGCCCTTCTT; | (SEQ ID NO: 71) |
| TCGTCGCTGTTGTCGTTTCTT; | (SEQ ID NO: 72) |
| TCCATGCGTGCGTGCGTTTT; | (SEQ ID NO: 73) |
| TCCATGCGTTGCGTTGCGTT; | (SEQ ID NO: 74) |
| TCCACGACGTTTTCGACGTT; | (SEQ ID NO: 75) |
| TCGTCGTTGTCGTTGTCGTT; | (SEQ ID NO: 76) |
| TCGTCGTTTTGTCGTTTTGTCGTT; | (SEQ ID NO: 77) |
| TCGTCGTTGTCGTTTTGTCGTT; | (SEQ ID NO: 78) |
| GCGTGCGTTGTCGTTGTCGTT; | (SEQ ID NO: 79) |
| TGTCGTTTGTCGTTTGTCGTT; | (SEQ ID NO: 80) |
| TGTCGTTGTCGTTGTCGTTGTCGTT; | (SEQ ID NO: 81) |
| TGTCGTTGTCGTTGTCGTT; | (SEQ ID NO: 82) |
| TCGTCGTCGTCGTT; | (SEQ ID NO: 83) |
| TGTCGTTGTCGTT; | (SEQ ID NO: 84) |
| TCCATAGCGTTCCTAGCGTT; | (SEQ ID NO: 85) |
| TCCATGACGTTCCTGACGTT; | (SEQ ID NO: 86) |
| GTCGYT; | (SEQ ID NO: 87) |
| TGTCGYT; | (SEQ ID NO: 88) |
| AGCTATGACGTTCCAAGG; | (SEQ ID NO: 89) |
| TCCATGACGTTCCTGACGTT; | (SEQ ID NO: 90) |
| ATCGACTCTCGAACGTTCTC; | (SEQ ID NO: 91) |
| TCCATGTCGGTCCTGACGCA; | (SEQ ID NO: 92) |
| TCTTCGAT; | (SEQ ID NO: 93) |
| ATAGGAGGTCCAACGTTCTC; | (SEQ ID NO: 94) |
| GCTAGAGGGGAGGGT; | (SEQ ID NO: 95) |
| GCTAGATGTTAGGGG; | (SEQ ID NO: 96) |
| GCTAGAGGGGAGGGT; | (SEQ ID NO: 97) |
| GCTAGAGGGGAGGGT; | (SEQ ID NO: 98) |
| GCATGAGGGGGAGCT; | (SEQ ID NO: 99) |
| ATGGAAGGTCCAGGGGGCTC; | (SEQ ID NO: 100) |
| ATGGACTCTGGAGGGGGCTC; | (SEQ ID NO: 101) |
| ATGGACTCTGGAGGGGGCTC; | (SEQ ID NO: 102) |
| ATGGACTCTGGAGGGGGCTC; | (SEQ ID NO: 103) |
| ATGGAAGGTCCAAGGGGCTC; | (SEQ ID NO: 104) |
| GAGAAGGGGGGACCTTCCAT; | (SEQ ID NO: 105) |
| GAGAAGGGGGGACCTTCGAT; | (SEQ ID NO: 106) |
| GAGAAGGGGGGACCTTGGAT; | (SEQ ID NO: 107) |
| GAGAAGGGGGGACCTTCCAT; | (SEQ ID NO: 108) |
| GAGAAGGGGGGACCTTCCAT; | (SEQ ID NO: 109) |
| GAGAAGGGGCCAGCACTGAT; | (SEQ ID NO: 110) |
| TCCATGTGGGGCCTGATGCT; | (SEQ ID NO: 111) |
| TCCATGTGGGGCCTGATGCT; | (SEQ ID NO: 112) |
| TCCATGAGGGGCCTGATGCT; | (SEQ ID NO: 113) |
| TCCATGTGGGGCCTGCTGAT; | (SEQ ID NO: 114) |
| ATGGACTCTCCGGGGTTCTC; | (SEQ ID NO: 115) |
| ATGGAAGGTCCGGGGTTCTC; | (SEQ ID NO: 116) |
| ATGGACTCTGGAGGGGTCTC; | (SEQ ID NO: 117) |
| ATGGAGGCTCCATGGGGCTC; | (SEQ ID NO: 118) |
| ATGGACTCTGGGGGGTTCTC; | (SEQ ID NO: 119) |
| ATGGACTCTGGGGGGTTCTC; | (SEQ ID NO: 120) |
| TGCATGTGGGTGGGGATGCT; | (SEQ ID NO: 121) |
| TCCATGCGGGTGGGGATGCT; | (SEQ ID NO: 122) |
| TCCATGGGGGTCCTGATGCT; | (SEQ ID NO: 123) |
| TCCATGGGGGTCCTGATGCT; | (SEQ ID NO: 124) |
| TCCATGTGGGGCCTGATGCT; | (SEQ ID NO: 125) |
| TCCATGTGGGGCCTGATGCT; | (SEQ ID NO: 126) |
| TCCATGGGGTCCCTGATGGT; | (SEQ ID NO: 127) |
| TCCATGGGGTGCCTGATGCT; | (SEQ ID NO: 128) |
| TCCATGGGGTTCCTGATGCT; | (SEQ ID NO: 129) |
| TCCATGGGGTCCCTGATGCT; | (SEQ ID NO: 130) |
| TCCATCGGGGGCCTGATGCT; | (SEQ ID NO: 131) |
| GCTAGAGGGAGTGT; | (SEQ ID NO: 132) |
| GGGGGGGGGGGGGGGGGGGG; | (SEQ ID NO: 133) |
| ACTGACAGACTGACAGACTGA; | (SEQ ID NO: 134) |
| AGTGACAGACAGACACACTGA; | (SEQ ID NO: 135) |
| ACTGACAGACTGATAGACCCA; | (SEQ ID NO: 136) |
| AGTGAGAGACTGCAAGACTGA; | (SEQ ID NO: 137) |
| AATGCCAGTCCGACAGGCTGA; | (SEQ ID NO: 138) |
| CCAGAACAGAAGCAATGGATG; | (SEQ ID NO: 139) |
| CCTGAACAGAAGCCATGGATG; | (SEQ ID NO: 140) |
| GCAGAACAGAAGACATGGATG; | (SEQ ID NO: 141) |
| CCACAACACAAGCAATGGATA; | (SEQ ID NO: 142) |
| AAGCTAGCCAGCTAGCTAGCA; | (SEQ ID NO: 143) |
| CAGCTAGCCACCTAGCTAGCA; | (SEQ ID NO: 144) |
| AAGCTAGGCAGCTAACTAGCA; | (SEQ ID NO: 145) |
| GAGCTAGCAAGCTAGCTAGGA; | (SEQ ID NO: 146) |

Nucleic acids having modified backbones, such as phosphorothioate backbones, also fall within the class of immunostimulatory nucleic acids. U.S. Pat. Nos. 5,723,335 and 5,663,153 issued to Hutcherson, et al. and related PCT publication WO95/26204 describe immune stimulation using phosphorothioate oligonucleotide analogues. These patents describe the ability of the phosphorothioate backbone to stimulate an immune response in a non-sequence specific manner. Thus some embodiments of the invention rely on the use of phosphorothioate backbone nucleic acids which lack CpG, poly-G and T-rich motifs.

In the case when the immunostimulatory nucleic acid is administered in conjunction with a nucleic acid vector, it is preferred that the backbone of the immunostimulatory nucleic acid be a chimeric combination of phosphodiester and phosphorothioate (or other phosphate modification). This is because the uptake of the plasmid vector by the cell may be hindered by the presence of completely phosphorothioate oligonucleotide. Thus when both a vector and an oligonucleotide are delivered to a subject, it is preferred that the oligonucleotide have a chimeric or phosphorothioate and that the plasmid be associated with a vehicle that delivers it directly into the cell, thus avoiding the need for cellular uptake. Such vehicles are known in the art and include, for example, liposomes and gene guns.

For use in the instant invention, the immunostimulatory nucleic acids can be synthesized de novo using any of a number of procedures well known in the art. Such compounds are referred to as “synthetic nucleic acids.” For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986, ; Garegg et al., Tet. Let. 27:4055-4058, 1986, Gaffney et al., Tet. Let. 29:2619-2622, 1988). These chemistries can be performed by a variety of automated oligonucleotide synthesizers available in the market. These nucleic acids are referred to as synthetic nucleic acids. Alternatively, immunostimulatory nucleic acids can be produced on a large scale in plasmids, (see Sambrook, T., et al., “Molecular Cloning: A Laboratory Manual”, Cold Spring Harbor laboratory Press, New York, 1989) and separated into smaller pieces or administered whole. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acids. The term “immunostimulatory nucleic acid” encompasses both synthetic and isolated immunostimulatory nucleic acids.

For use in vivo, nucleic acids are preferably relatively resistant to degradation (e.g., are stabilized). A “stabilized nucleic acid molecule” shall mean a nucleic acid molecule that is relatively resistant to in vivo degradation (e.g. via an exo- or endo-nuclease). Stabilization can be a function of length or secondary structure. Immunostimulatory nucleic acids that are tens to hundreds of kbs long are relatively resistant to in vivo degradation. For shorter immunostimulatory nucleic acids, secondary structure can stabilize and increase their effect. For example, if the 3′ end of a nucleic acid has self-complementarity to an upstream region, so that it can fold back and form a sort of stem loop structure, then the nucleic acid becomes stabilized and therefore exhibits more biological in vivo activity.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. One type of modified backbone is a phosphate backbone modification. Immunostimulatory nucleic acids, including at least two phosphorothioate linkages at the 5′ end of the oligonucleotide and multiple phosphorothioate linkages at the 3′ end, preferably 5, can in some circumstances provide maximal activity and protect the nucleic acid from degradation by intracellular exo- and endo-nucleases. Other phosphate modified nucleic acids include phosphodiester modified nucleic acids, combinations of phosphodiester and phosphorothioate nucleic acids, methylphosphonate, methylphosphorothioate, phosphorodithioate, and combinations thereof. Each of these combinations in CpG nucleic acids and their particular effects on immune cells is discussed in more detail in PCT Published Patent Applications PCT/US95/01570 and PCT/US97/19791, the entire contents of which are hereby incorporated by reference. Although not intending to be bound by any particular theory, it is believed that these phosphate modified nucleic acids may show more stimulatory activity due to enhanced nuclease resistance, increased cellular uptake, increased protein binding, and/or altered intracellular localization.

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863. Alkylphosphotriesters, in which the charged oxygen moiety is alkylated as described in U.S. Pat. No. 5,023,243 and European Patent No. 092,574, can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Both phosphorothioate and phosphodiester nucleic acids containing immunostimulatory motifs are active in immune cells. However, based on the concentration needed to induce immunostimulatory nucleic acid specific effects, the nuclease resistant phosphorothioate backbone immunostimulatory nucleic acids are more potent than phosphodiester backbone immunostimulatory nucleic acids. For example, 2 μg/ml of the phosphorothioate has been shown to effect the same immune stimulation as a 90 μg/ml of the phosphodiester.

Another type of modified backbone, useful according to the invention, is a peptide nucleic acid. The backbone is composed of aminoethylglycine and supports bases which provide the DNA character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. Peptide nucleic acids can be purchased from various commercial sources, e.g., Perkin Elmer, or synthesized de novo.

Another class of backbone modifications include 2′-O-methylribonucleosides (2′-Ome). These types of substitutions are described extensively in the prior art and in particular with respect to their immunostimulating properties in Zhao et al., Bioorganic and Medicinal Chemistry Letters, 1999, 9:24:3453. Zhao et al. describes methods of preparing 2′-Ome modifications to nucleic acids.

The nucleic acid molecules of the invention may include naturally-occurring or synthetic purine or pyrimidine heterocyclic bases as well as modified backbones. Purine or pyrimidine heterocyclic bases include, but are not limited to, adenine, guanine, cytosine, thymidine, uracil, and inosine. Other representative heterocyclic bases are disclosed in U.S. Pat. No. 3,687,808, issued to Merigan, et al. The terms “purines” or “pyrimidines” or “bases” are used herein to refer to both naturally-occurring or synthetic purines, pyrimidines or bases.

Other stabilized nucleic acids include non-ionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The immunostimulatory nucleic acids having backbone modifications useful according to the invention in some embodiments are S- or R-chiral immunostimulatory nucleic acids. An “S chiral immunostimulatory nucleic acid” as used herein is an immunostimulatory nucleic acid wherein at least two nucleotides have a backbone modification forming a chiral center and wherein a plurality of the chiral centers have S chirality. An “R chiral immunostimulatory nucleic acid” as used herein is an immunostimulatory nucleic acid wherein at least two nucleotides have a backbone modification forming a chiral center and wherein a plurality of the chiral centers have R chirality. The backbone modification may be any type of modification that forms a chiral center. The modifications include but are not limited to phosphorothioate, methylphosphonate, methylphosphorothioate, phosphorodithioate, 2′-Ome and combinations thereof.

The chiral immunostimulatory nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), a plurality have a single chirality, S or R. A “plurality” as used herein refers to an amount greater than 50%. Thus, less than all of the chiral centers may have S or R chirality as long as a plurality of the chiral centers have S or R chirality. In some embodiments at least 55%, 60%, 65%, 70%, 75%, 80,%, 85%, 90%, 95%, or 100% of the chiral centers have S or R chirality. In other embodiments at least 55%, 60%, 65%, 70%, 75%, 80%, 85%, 90%, 95%, or 100% of the nucleotides have backbone modifications.

The S- and R-chiral immunostimulatory nucleic acids may be prepared by any method known in the art for producing chirally pure oligonucleotides. Stec et al teach methods for producing stereopure phosphorothioate oligodeoxynucleotides using an oxathiaphospholane. (Stec, W. J., et al., 1995, J. Am. Chem. Soc., 117:12019). Other methods for making chirally pure oligonucleotides have been described by companies such as ISIS Pharmaceuticals. US Patents which disclose methods for generating stereopure oligonucleotides include U.S. Pat. Nos. 5,883,237, 5,837,856, 5,599,797, 5,512,668, 5,856,465, 5,359,052, 5,506,212, 5,521,302 and 5,212,295, each of which is hereby incorporated by reference in its entirety.

As used herein, administration of an immunostimulatory nucleic acid is intended to embrace the administration of one or more immunostimulatory nucleic acids which may or may not differ in terms of their profile, sequence, backbone modifications and biological effect. As an example, CpG nucleic acids and poly-G nucleic acids may be administered to a single subject along with a cancer medicament. In another example, a plurality of CpG nucleic acids which differ in nucleotide sequence may also be administered to a subject.

The invention in one aspect encompasses the administration of the immunostimulatory nucleic acids along with a cancer medicament in order to provide a synergistic effect useful in the prevention and/or treatment of cancer. The beneficial effects of the immunostimulatory nucleic acids is due, in part, to the modulation and stimulation of Th1 and/or Th2 immune responses by these nucleic acids. The immunostimulatory nucleic acids of the invention may provide the synergistic response via a number of mechanisms, including but not limited to stimulation of hemopoietic recovery during or following cancer therapy, anti-microbial infection activity, enhancement of uptake of cancer medicaments by cancer cells or immune cells (depending upon the nature of the cancer medicament), and inhibition or prevention of allergic responses to cancer medicament. In some instances, Th1 responses will be most effective, particularly in fighting bacterial, viral or fungal infection. Th1 responses will also be most useful when a diminution of an allergic response to transfusion by-products is required. In other instances, a Th2 response will be most beneficial. In still other instances, a plurality of immunostimulatory nucleic acids may be administered, the plurality having at least one immunostimulatory nucleic acid which induces a Th1 response and at least one immunostimulatory nucleic acid which induces a Th2 response.

Immunostimulatory nucleic acids may function by enhancing the recovery of marrow cells following chemotherapy, or radiation. It is often the case that subsequent rounds of anti-cancer therapy are delayed until the patient's marrow has recovered sufficiently to provide an adequate number of erythrocytes, neutrophils and platelets, to ensure the hemopoietic survival of the patient. The nucleic acids accelerate this recovery, and thus allow for more frequent and, in some cases, higher dosed administration of the cancer medicament. Additionally, the ability of the nucleic acids to stimulate marrow recovery, through the proliferation and/or differentiation of hemopoietic precursors, prevents some unwanted side effects of cancer medicaments including weakness, uncontrolled bleeding, and susceptibility to infection due to reduced numbers of erythrocytes, platelets and neutrophils, respectively.

The immunostimulatory nucleic acids function to enhance defense mechanisms against bacterial, fungal, parasitic and viral infections. The prevention and control of such infections in immunocompromised cancer patients is a major challenge in the treatment and management of the disease. Such infections can usually disadvantageously delay or alter the course of treatment for cancer patients. The cellular and humoral immune responses stimulated by the nucleic acids reflect the body's own natural defense system against invading pathogens. The immunostimulatory nucleic acids perform this function through the activation of innate immunity which is known to be most effective in the elimination of microbial infections. Enhancement of innate immunity occurs, inter alia, via increased IFN-α production and increased NK cell activity, both of which are effective in the treatment of microbial infections. The immunostimulatory nucleic acids also function by enhancement of antibody-dependent cell cytotoxicity. This latter mechanism provides long-lasting effects of the nucleic acids, thereby reducing dosing regimes, improving compliance and maintenance therapy, reducing emergency situations; and improving quality of life. Some examples of common opportunistic infections in cancer patients are caused by Listeria monocytogenes, Pneumocystis carinii, cytomegalovirus, Mycobacterium tuberculosis, Staphylococcus aureus, Streptococcus pneumoniae, Haemophilus influenzae, Escherichia coli, Klebsiella pneumoniae, Pseudomonas aeruginosa, Nocardia, Candida, Aspergillus, and herpes viruses such as herpes simplex virus.

Enhanced uptake of cancer medicaments (e.g., doxorubicin, mephalan) by cancer cells or immune cells is another way in which immunostimulatory nucleic acids function in the treatment of cancer. Although not intending to be bound by any particular theory, it is possible that immunostimulatory nucleic acids enhance uptake by inducing the release of a multitude of cytokines including TNF-α. Surprisingly, it has been found according to the invention that uptake of cancer medicaments can be effected even without conjugation of the immunostimulatory nucleic acid to the cancer medicament.

It is sometimes the case that subjects undergoing cancer treatment experience an adverse allergic reaction to the cancer medicament formulation being administered. The reaction may be specific to the cancer medicament itself or to other substances included in the cancer medicament formulation (e.g., the carrier substance, stabilizing agents, or sterilizing agents within the formulation). An example of a medicament which often triggers an allergic reaction upon administration is a formulation of taxol. This allergic reaction makes the use of such a medicament less desirable, and at the very least, may lead to the administration of the medicament at lower than therapeutic doses in order to avoid the allergic reaction. The present invention provides a method for avoiding such an adverse reaction through the administration of an immunostimulatory nucleic acid. In preferred embodiments, the immunostimulatory nucleic acid is one which minimizes or altogether inhibits a Th2 immune response. Th2 immune response are associated with allergic reactions. Thus, by suppressing Th2 reactions as can be accomplished through the administration of some of the immunostimulatory nucleic acids of the invention, the allergic reaction associated with some cancer medicaments and/or their particular formulations, can be avoided. For example, since CpG immunostimulatory nucleic acids function not only to elicit a Th1 response but also to suppress Th2 responses, the subject may be administered a CpG immunostimulatory nucleic acid prior to or at the time of the administration of the cancer medicament in order to prevent or diminish the Th2 allergic reaction which might otherwise occur. In an important embodiment, Th2 suppressing immunostimulatory nucleic acids are administered with the cancer medicament taxol. Reducing or eliminating the allergic reaction altogether may also allow for administration of cancer medicaments in doses greater than the therapeutic dose, or at least greater than the doses currently administered.

The immunostimulatory nucleic acids of the invention are also useful in the regulation of adverse allergic reactions in subjects undergoing transfusions. Subjects undergoing cancer treatment often require transfusions of red cells and/or platelets. Either due to incomplete separation of these cell types from others or due to differences in minor histocompatibility loci between the donor and the recipient of these blood products, subjects being infused may experience an acute allergic reaction to the transfusion. To counter this reaction which is primarily a Th2 type response, patients are administered allergy medication such as anti-histamines. Since CpG and T-rich immunostimulatory nucleic acids function not only to elicit a Th1 response but also to suppress Th2 responses, the subject may be administered a CpG or a T-rich immunostimulatory nucleic acid prior to or at the time of the transfusion in order to prevent or diminish the Th2 allergic reaction which might otherwise occur. Other immunostimulatory nucleic acids may be used for this same purpose in addition to CpG and T-rich immunostimulatory nucleic acids.

Table 2 lists a number of benefits resulting from the combined use of immunostimulatory nucleic acids and cancer medicaments.

TABLE 2

| Differentiating Product Features | Benefits |
|---|---|
| Induces potent, Th1-type immune activation and | Breakthrough therapeutic capability that enables |
| particularly strong cellular immune stimulation, | cancer immunotherapies, increases response rates, |
| enhances ADCC, IFNα, NK activity, DC activity | increases long-term survival, may help expand |
| | indications and treatable populations |
| Provides additional anticancer activity via NK cell | Further enhances efficacy |
| activation, IFNα production | |
| Produces systemic effects in the body | Can be used to treat metastatic tumors |
| Promotes antigen-specific immune responses | Specifically targeted immune responses that do not |
| | harm normal tissues |
| Induces hematopoiesis | Accelerates bone marrow recovery, immune system |
| | function in immunocompromised patients |
| Upregulates innate immunity; provides early | Provides immediate, broad protection against |
| antiinfective activity | infectious pathogens |
| Enhances efficacy and decreases side-effects of | Permits increases in maximum tolerable doses, |
| chemotherapies and combination (chemo + immuno) | provides additional anticancer activity, enhances |
| therapies | MAb/Ag activity in combination therapies, reduces |
| | neutropenia and myelosuppression, decreases |
| | infectious episodes |
| Effective SC, IN, ID, IM, IP, IV or oral delivery | Multiple formulations, modes of delivery possible |

A cancer cell is a cell that divides and reproduces abnormally due to a loss of normal growth control. Cancer cells almost always arise from at least one genetic mutation. In some instances, it is possible to distinguish cancer cells from their normal counterparts based on profiles of expressed genes and proteins, as well as to the level of their expression. Genes commonly affected in cancer cells include oncogenes, such as ras, neu/HER2/erbB, myb, myc and abl, as well as tumor suppressor genes such as p53, Rb, DCC, RET and WT. Cancer-related mutations in some of these genes leads to a decrease in their expression or a complete deletion. In others, mutations cause an increase in expression or the expression of an activated variant of the normal counterpart. Genetic mutations in cancer cells can be targets of cancer medicaments in some instances. For example, some medicaments target proteins which are thought to be necessary for cancer cell survival and division, such as cell cycle proteins (e.g., cyclin dependent kinases), telomerase and telomerase associated proteins, and tumor suppressor proteins, many of which are upregulated, or unregulated, in cancer cells.

The term “tumor” is usually equated with neoplasm, which literally means “new growth” and is used interchangeably with “cancer.” A “neoplastic disorder” is any disorder associated with cell proliferation, specifically with a neoplasm. A “neoplasm” is an abnormal mass of tissue that persists and proliferates after withdrawal of the carcinogenic factor that initiated its appearance. There are two types of neoplasms, benign and malignant. Nearly all benign tumors are encapsulated and are noninvasive; in contrast, malignant tumors are almost never encapsulated but invade adjacent tissue by infiltrative destructive growth. This infiltrative growth can be followed by tumor cells implanting at sites discontinuous with the original tumor. The method of the invention can be used to treat neoplastic disorders in humans, including but not limited to: sarcoma, carcinoma, fibroma, leukemia, lymphoma, melanoma, myeloma, neuroblastoma, rhabdomyosarcoma, retinoblastoma, and glioma as well as each of the other tumors described herein.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

The immunostimulatory nucleic acids are useful for treating or preventing cancer or cancer in a subject. A “subject” shall mean a human or vertebrate mammal including but not limited to a dog, cat, horse, cow, pig, sheep, goat, or primate, e.g., monkey. The invention can also be used to treat cancer and tumors in non human subjects. Cancer is one of the leading causes of death in companion animals (i.e., cats and dogs). Cancer usually strikes older animals which, in the case of house pets, have become integrated into the family. Forty-five % of dogs older than 10 years of age, are likely to succumb to the disease. The most common treatment options include surgery, chemotherapy and radiation therapy. Others treatment modalities which have been used with some success are laser therapy, cryotherapy, hyperthermia and immunotherapy. The choice of treatment depends on type of cancer and degree of dissemination. Unless the malignant growth is confined to a discrete area in the body, it is difficult to remove only malignant tissue without also affecting normal cells.

Malignant disorders commonly diagnosed in dogs and cats include but are not limited to lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma. Other neoplasias in dogs include genital squamous cell carcinoma, transmissable veneral tumor, testicular tumor, seminoma, Sertoli cell tumor, hemangiopericytoma, histiocytoma, chloroma (granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma. Additional malignancies diagnosed in cats include follicular lymphoma, intestinal lymphosarcoma, fibrosarcoma and pulmonary squamous cell carcinoma. The ferret, an ever-more popular house pet, is known to develop insulinoma, lymphoma, sarcoma, neuroma, pancreatic islet cell tumor, gastric MALT lymphoma and gastric adenocarcinoma.

Neoplasias affecting agricultural livestock include leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lumphadenitis (CLA): chronic, infectious, contagious disease of sheep and goats caused by the bacterium Corynebacterium pseudotuberculosis, and contagious lung tumor of sheep caused by jaagsiekte.

In one aspect, a method for treating cancer is provided which involves administering the compositions of the invention to a subject having cancer. A “subject having cancer” is a subject that has been diagnosed with a cancer. In some embodiments, the subject has a cancer type characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue.

However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

With respect to the prophylactic treatment methods, the invention is aimed at administering the compositions of the invention to a subject at risk of developing cancer. A subject at risk of developing a cancer is one who has a high probability of developing cancer. These subjects include, for instance, subjects having a genetic abnormality, the presence of which has been demonstrated to have a correlative relation to a higher likelihood of developing a cancer. Subjects exposed to cancer causing agents such as tobacco, asbestos, or other chemical toxins are also subjects at risk of developing cancers used herein. When a subject at risk of developing a cancer is treated with an immunostimulatory nucleic acid and a cancer medicament, such as a cancer vaccine in the form of a cancer antigen, on a regular basis, such as monthly, the subject will be able to recognize and produce an antigen specific immune response. If a tumor begins to form in the subject, the subject will develop a specific immune response against one or more of the cancer antigens. This aspect of the invention is particularly advantageous when the antigen to which the subject will be exposed is known. For instance, subjects employed in certain trades which are exposed to cancer-causing agents on an ongoing basis would be ideal subjects for treatment according to the invention, particularly because cancer-causing agents usually preferentially target a specific organ or tissue. For example, many air borne, or inhaled, carcinogens such as tobacco smoke and asbestos have been associated with lung cancer. The methods in which a subject is passively exposed to an carcinogen can be particularly dependent on timing of the administration of the immunostimulatory nucleic acid and the cancer medicament, preferably in the form of a cancer vaccine (e.g., a cancer antigen). For instance, in a subject at risk of developing a cancer, the subject may be administered the immunostimulatory nucleic acid and the cancer vaccine containing a cancer antigen on a regular basis when that risk is greatest, i.e., after exposure to a cancer causing agent.

A carcinogen is an agent capable of initiating development of malignant cancers. Exposure to carcinogens generally increase the risk of neoplasms in subjects, usually by affecting DNA directly. Carcinogens may take one of several forms such as chemical, electromagnetic radiation, or may be an inert solid body.

Substances for which there is sufficient evidence to establish a causal relationship in cancer in humans are referred to as confirmed human carcinogens. Included in this category are the following substances: Aflatoxins, Alcoholic beverages, Aluminium production, 4-aminobiphenyl, Arsenic and arsenic compounds, Asbestos, Manufacture of auramine, Azathioprine, Benzene, Benzidine, Beryllium and compounds, Betel quid with tobacco, Bis(chloromethyl)ether and chloromethyl methyl ether (technical grade), Boot and shoe manufacture and repair (occupational exposure), 1,4 Butanediol dimethanesulphonate (Myleran), Cadmium and compounds, Chlorambucil, Chlornaphazine, 1-(2-Chloroethyl)-3-(4-methylcyclohexyl)-1 nitrosourea, Chloromethyl methyl ether (technical), Chromium compounds (hexavalent), Coal gasification, Coal tar pitches, Coal tars, Coke production, Cyclophosphamide, Cyclosporin, Erionite, Ethylene oxide, Furniture and cabinet making, Underground haematite mining with exposure to radon, Iron and steel founding, Isopropyl alcohol manufacture (strong acid process), Manufacture of magenta, Melphalan, 8-Methoxypsoralen (Methoxsalen) plus ultraviolet radiation, Mineral oils-untreated and mildly-treated oils, MOPP and other combined chemotherapy for cancer, Mustard gas (sulphur mustard), 2-Naphthylamine, Nickel and nickel compounds (essentially sulphate and sulphide), Nonsteroidal oestrogens (not necessarily all in group) includes diethylstilboestrol, Oestrogen replacement therapy, and Combined oral contraceptives and sequential oral contraceptives, Steroidal oestrogens (not all in group), Painter (occupational exposure as a painter), Phenacetin (analgesic mixtures containing), Rubber industry, Salted fish (Chinese style), Solar radiation, Shale oils, Soots, Sulphuric acid (occupational exposures to strong-inorganic-acid mists of sulphuric acid), Talc containing asbestiform fibres, Thiotepa, Tobacco products (smokeless), Tobacco smoke, Treosulphan, and Vinyl chloride.

Substances for which there is a lesser degree of evidence in humans but sufficient evidence in animal studies, or degrees of evidence considered unequivocal of mutagenicity in mammalian cells are referred to as probable human carcinogens. This category of substances includes: Acrylamide, Acrylonitrile, Adriamycin, Anabolic steroids, Azacitidine, Benzanthracene, Benzidine-based dyes (technical grade), Direct Black 38, Direct Blue 6, Direct Brown 95, Benzopyrene1,3-Butadiene, Captafol, Bischloroethyl nitrosourea (BCNU), 1-(2-Chloroethyl)-3-cyclohexyl-1-nitrosourea (CCNU), Chloramphenicolpara-Chloro-ortho-toluidine and its strong acid salts, Chlorozotocin, Cisplatin, Creosotes, Dibenzanthracene, Diesel engine exhaust, Diethyl sulphate, Dimethylcarbamoyl chloride, Dimethyl sulphate, Epichlorohydrin, Ethylene dibromide, N-ethyl-N-nitrosourea, Formaldehyde, Glass manufacturing industry (occupational exposure), Art glass (glass containers and pressed ware), Hairdresser or barber (occupational exposure, probably dyes), Insecticide use (occupational), IQ (2-Amino-3-methylimidazo[4,5-f]quinoline), Mate drinking (hot), 5-Methoxypsoralen, 4,4′-Methylenebis(2-chloroaniline) (MOCA), N-Methyl-N-nitro-N-nitrosoguanidine (MNNG), N-Methyl-N-nitrosourea, Nitrogen mustard, N-Nitrosodiethylamine, N-Nitrosodimethylamine, Petroleum refining (occupational refining exposures), Phenacetin, Polychlorinated biphenyls, Procarbazine hydrochloride, Silica (crystalline), Styrene-7,8-oxide, Tris(1-azaridinyl)phosphine sulphide (Thiotepa), Tris(2,3-dibromopropyl)phosphate, Ultraviolet radiation: A, B and C including sunlamps and sunbeds, and Vinyl bromide.

Substances for which there is sufficient evidence in animal tests are referred to as possible human carcinogens. This category of substances includes: A-C(2-Amino-9H-pyrido[2,3-b]indole), Acetaldehyde, Acetamide, AF-2[2-(2-Furyl)-3-(5-nitro-2-furyl)acrylamide, para-Aminoazobenzene, ortho-Aminoazobenzene, 2-Amino-5-(5-nitro-2-furyl)-1,3,4-thiadiazole, Amitrole, ortho-Anisidine, Antimony trioxide, Aramite, Atrazine, Attapulgite, Azaserine, Benzo[b]fluoranthene, Benzo[j]fluoranthene, Benzo[k]fluoranthene, Benzyl violet, Bitumens (extracts of steam-refined and air-refined bitumens), Bleomycins, Bracken ferns, Bromodichloromethane, Butylated hydroxyanisole (BHA), á-Butyrolactone, Caffeic acid, Carbon black extract, Carbon tetrachloride, Carrageenan (degraded), Ceramic fibres, Chloramphenicol, Chlordane, Chlordecone, Chlorendic acid, Chlorinated paraffins of average carbon-chain length C12 and average degree of chlorination approx 60%, alpha-Chlorinated toluenes (not necessarily all in group), Benzotrichloride, para-Chloroaniline, Chloroform, Chlorophenols, Pentachlorophenol, 2,4,6-Trichlorophenol, Chlorophenoxy herbicides (not necessarily all in group), 4-Chloro-ortho-phenylenediamine, CI Acid Red 114, CI Basic Red 9, CI Direct Blue 15, Citrus Red No.2, Cobalt and cobalt compounds, Coffee (bladder), para-Cresidine, Cycasin, Dacarbazine, Dantron (1,8-dihydroxyanthraquinone), Daunomycin, DDT, N,N′-Diacetylbenzidine, 4,4′-Diaminodiphenyl ether, 2,4-Diaminotoluene, Dibenz[a,h]acridine, Dibenz[a,j]acridine, 7H-Dibenzo[c,g]carbazole, Dibenzo[a,e]pyrene, Dibenzo[a,h]pyrene, Dibenzo[ai]pyrene, Dibenzo[a,l]pyrene, 1,2-Dibromo-3-chloropropane, para-Dichlorobenzene, 3,3′-Dichlorobenzene, 3,3′-Dichloro-4,4′-diaminodiphenyl ether, 1,2-Dichloroethane, Dichloromethane, 1,3-Dichloropropene (technical grade), Dichlorvos, Diepoxybutane, Diesel fuel (marine), Di(2-ethylhexyl)phthalate, 1,2-Diethylhydrazine, Diglycidyl resorcinol ether, Dihydrosafrole, Diisopropyl sulfate, 3,3′-Dimethoxybenzidine, para-Dimethylaminoazobenzene, trans-2-[(Dimethylamino)methylimino]-5-[2-(5-nitro-2-furyl[vinyl]-1,3,4-oxidiazole, 2,6-Dimethylaniline (2,6-Xylidene), 3,3′-Dimethylbenzidine (ortho-tolidine), Dimethylformamide, 1,1-Dimethylhydrazine, 1,2-Dimethylhydrazine, 1,6-Dinitropyrene, 1,8-Dinitropyrene, 1,4-Dioxane, Disperse Blue 1Ethyl acrylateEthylene thioureaEthyl methanesulphonate 2-(2-Formylhydrazino)-4-(5-nitro-2-furyl)thiazoleFuel oils (residual, heavy) Fusarium moniliforme (toxins derived from) Fumonisin B1; Fumonisin B2; Fusarin C, Gasoline, Gasoline engine exhausts, Glasswool, Glu-P-1 (2-Amino-6-methyldipyrido[1,2-a: 3′2′-d]imidazole), Glu-P-2(-Aminodipyrido[1,2-a:3′2′-d]imidazole), Glycidaldehyde, Griseofulvin, HC Blue No 1, Heptachlor, Hexachlorobenzene, Hexachlorocyclohexanes Technical grades alpha isomer gamma isomer (lindane), Hexamethylphosphoramide, Hydrazine, Indeno[1,2,3-cd]pyrenelron-dextran complex, Isoprene, Lasiocarpine, Lead and lead compounds (inorganic), Magenta (containing CI Basic Red 9), Man-made mineral fibres (see glasswool, rockwool, slagwool, and ceramic fibres), MeA-a-C (2-Amino-3-methyl-9H-pyrido[2,3-b]indole), MeIQ (2-Amino-3,4-dimethylimidazo[4,5-f]-quinolone), MeIQx (2-Amino-3,8-dimethylamidazo[4,5-f]quinoxaline), Methylmercury compounds (methylmercuric chloride), Merphalan, 2-Methylaziridine, Methylazoxymethanol and its acetate, 5-Methylchrysene, 4,4′-Methylenebis(2-methylaniline), 4,4′-Methylenedianiline, Methylmethanesulphonate, 2-methyl-1-nitroanthraquinone (uncertain purity), N-methyl-N-nitrosourethane, Methylthiouracil, Metronidazole, Mirex, Mitomycin, Monocrotaline 5-(Morpholinomethyl)-3-[(5-nitrofurfurylidene)amino]-2-oxazolidinone, Nafenopin, Niridazole, 5-Nitroacenaphthene, 6-Nitrochrysene, Nitrofen (technical grade), 2-Nitrofluorene1-[(5-Nitrofurfurylidene)amino]-2-imidazolidinone, N-[4-(5 -Nitro-2-furyl)-2-thiazolyl]acetamide, Nitrogen mustard, N-oxide, Nitrolotriacetic acid and its salts, 2-Nitropropane1-Nitropyrene, 4-Nitropyrene, N-Nitrosodi-n-butylamine, N-Nitrosodiethanolamine, N-Nitrosodi-n-propylamine, 3-(N-Nitrosomethylamino)propionitrile, 4-(N-Nitrosomethylamino)-1-(3-pyridyl)-1-butanone (NNK), N-Nitrosomethylethylamine, N-Nitrosomethylvinylamine, N-Nitrosomorpholine, N-Nitrosonornicotine, N-Nitrosopiperidene, N-Nitrosopyrrolidine, N-Nitrososarcosine, Ochratoxin A, Oil Orange, Panfuran S (containing dihydroxymethylfuratzine), Phenazopyridine hydrochloride, Phenobarbital, Phenoxybenzamine hydrochloride, Phenyl glycidyl ether, PhenytoinPhIP (2-Amino-1-methyl-6-phenylimidazo[4,5-b]pyridine, Pickled vegetables, traditional Asian, Polybrominated biphenyls, Ponceau MXPonceau 3R, Potassium bromate, 1,3-Propane sultone, Propylene oxide, Progestins, Medroxyprogesterone acetate, á-Propiolactone, Propylthiouracil, Rockwool, SaccharinSafroleSlagwoolSodium ortho-phenylphenate, Sterigmatocystin, Streptozotocin, Styrene, Sulfallate, 2,3,7,8-Tetrachlorodibenzo-para-dioxin (TCDD), Tetrachloroethylene, Textile manufacturing (occupational exposures), Thiocetamide, 4,4′-Thiodianiline, Thiourea, Toluene, diisocyanatesortho-Toluidine, Toxaphene (polychlorinated camphenes), Trichlormethine (trimustine hydrochloride), Trp-P-1 (3-Amino-1,4-dimethyl-5-H-pyrido[4,3-b]indole), Trp-P-2 (3-Amino-1-methyl-5H-pyrido[4,3-b]indole), Trypan blue, Uracil mustard, Urethane, 4-Vinylcyclohexene, 4-Vinylcyclohexene diepoxide, Welding fumes, Wood industries and Carpentry and joinery.

Subjects at risk of developing cancer also include those who have a genetic predisposition to cancer. In many cases, genetic predispositions to cancer can be identified by studying the occurrence of cancer in family members. Examples of genetic predisposition to common forms of cancer include, but are not limited to, mutation of BRCA1 and BRCA2 in familial breast cancer, mutation of APC in familial colony cancer (familial polyposis coli), mutation of MSH2 and MLH1 in hereditary nonpolyposis colon cancer (HNPCC), mutation of p53 in Li-Fraumeni syndrome, mutation of Rb1 in retinoblastoma, mutation of RET in multiple endocrine neoplasia type 2 (MEN2), mutation of VHL in renal cancer and mutation of WT1 in Wilm's tumor. Other cancers for which a familial predisposition has been identified include ovarian, prostate, melanoma and lung cancer.

It has been estimated that almost half of all currently diagnosed cancers will be treated with some form of cancer medicament. However, many forms of cancer, including melanoma, colorectal, prostate, endometrial, cervical and bladder cancer, do not respond well to treatment with cancer medicaments. In fact, only about 5-10 percent of cancers can be cured using cancer medicaments alone. These include some forms of leukemias and lymphomas, testicular cancer, choriocarcinoma, Wilm's Tumor, Ewing's sarcoma, neuroblastoma, small-cell lung cancer and ovarian cancer. Treatment of still other cancers, including breast cancer, requires a combination therapy of surgery or radiotherapy in conjunction with a cancer medicament.

Table 3 summarizes some of the current conventional treatment strategies for a broad range of cancers.

TABLE 3

| Cancer Type | Current Therapy |
|---|---|
| Bladder | Surgery, radiation & combo chemotherapy (MTX, Vincristine, Adriamycin |
| | & Cisplatin) |
| Brain | Surgery, radiation & chemotherapy (non-sugar containing |
| | chloroethylnitrosoureas |
| Breast | Surgery, radiation, chemotherapy & hormone therapy (Tamoxifen) |
| Cervical | Surgery, radiation & combo chemotherapy (Adriamycin & MTX) - not |
| | common |
| Colorectal | Surgery, radiation & chemotherapy (5-FU) |
| Esophagus | Surgery, radiation & chemotherapy (5-FU, Cisplatin, Bleomycin, |
| | Mitomycin C, Adriamycin & MTX) |
| Kidney | Surgery & radiation (chemotherapy ineffective) |
| Leukemia | Radiation & chemotherapy |
| Liver | Surgery, radiation & chemotherapy (Doxorubicin & Cisplatin) |
| Lung | SCLC: Chemotherapy +/− radiation NSCLC: Surgery & chemotherapy |
| Lymphoma | Radiation & chemotherapy (dependent on type of lymphoma) |
| Melanoma | Surgery, radiation & chemotherapy (Dacarbazine & Nitrosoureas) - not |
| | effective |
| Multiple Myeloma | Chemotherapy (Alkylating agents esp. Melphelan & Cyclophosphamide) + Prednisone |
| Oral/Pharyngeal | Surgery & radiation |
| Ovarian | Surgery & chemotherapy (Alkylating agents) |
| Pancreas | Surgery, radiation, chemotherapy (5-FU) |
| Prostate | Surgery, radiation, chemotherapy & hormonal therapy LHRH analogs, anti- |
| | androgens |
| Stomach | Surgery, radiation & combo chemotherapy FAM, FAMe, FAP, ELF |
| Uterus | Surgery, radiation, chemotherapy & hormonal therapy (Progesterone & |
| | Tamoxifen) |

As used herein, a “cancer medicament” refers to a agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. Cancer medicaments embrace such categories as chemotherapeutic agents, immunotherapeutic agents, cancer vaccines, hormone therapy, and biological response modifiers. Cancer medicaments also include agents which are administered to a subject in order to reduce the symptoms of a cancer, rather than to reduce the tumor or cancer burden (i.e., the number of cancer or tumor cells) in a subject. One example of this latter type of cancer medicament is a blood transfusion which is administered to a subject having cancer in order to maintain red blood cell and/or platelet levels within a normal range. As an example, in the absence of such transfusion, cancer patients with below normal levels of platelets are at risk of uncontrolled bleeding.

A cancer medicament does not refer to either surgical procedures or radiotherapy aimed at treating cancer. According to various aspects of the invention, some forms of immunostimulatory nucleic acids (e.g., poly G or CpG) and a cancer medicament may be administered after a surgical procedure and/or radiation therapy aimed at treating a cancer. Surgery and radiation are still commonly used to treat a variety of cancers, as shown in Table 2. In some cases, surgery is also used in a prophylactic manner to reduce the risk that a cancer will develop. As an example of this latter use of surgery, subjects at risk of developing breast cancer, for example, those with a familial disposition to breast cancer, sometimes undergo surgical breast removal (i.e., a mastectomy), in order to reduce the risk of developing the disease. Thus, a subject at risk of developing a cancer, such as breast cancer, can be treated according to the methods of the invention, with surgery followed by the administration of an immunostimulatory nucleic acid and a cancer medicament. Additionally, the methods of the invention are intended to embrace the use of more than one cancer medicament along with the immunostimulatory nucleic acids. As an example, where appropriate, the immunostimulatory nucleic acids may be administered with a both a chemotherapeutic agent and an immunotherapeutic agent. Alternatively, the cancer medicament may embrace an immunotherapeutic agent and a cancer vaccine, or a chemotherapeutic agent and a cancer vaccine, or a chemotherapeutic agent, an immunotherapeutic agent and a cancer vaccine all administered to one subject for the purpose of treating a subject having a cancer or at risk of developing a cancer.

Cancer medicaments function in a variety of ways. Some cancer medicaments work by targeting physiological mechanisms that are specific to tumor cells. Examples include the targeting of specific genes and their gene products (i.e., proteins primarily) which are mutated in cancers. Such genes include but are not limited to oncogenes (e.g., Ras, Her2, bcl-2), tumor suppressor genes (e.g., EGF, p53, Rb), and cell cycle targets (e.g., CDK4, p21, telomerase). Cancer medicaments can alternately target signal transduction pathways and molecular mechanisms which are altered in cancer cells. Targeting of cancer cells via the epitopes expressed on their cell surface is accomplished through the use of monoclonal antibodies. This latter type of cancer medicament is generally referred to herein as immunotherapy.

Other cancer medicaments target cells other than cancer cells. For example, some medicaments prime the immune system to attack tumor cells (i.e., cancer vaccines). Still other medicaments, called angiogenesis inhibitors, function by attacking the blood supply of solid tumors. Since the most malignant cancers are able to metastasize (i.e., exist the primary tumor site and seed a distal tissue, thereby forming a secondary tumor), medicaments that impede this metastasis are also useful in the treatment of cancer. Angiogenic mediators include basic FGF, VEGF, angiopoietins, angiostatin, endostatin, TNFα, TNP-470, thrombospondin-1, platelet factor 4, CAI, and certain members of the integrin family of proteins. One category of this type of medicament is a metalloproteinase inhibitor, which inhibits the enzymes used by the cancer cells to exist the primary tumor site and extravasate into another tissue.

Some cancer cells are antigenic and thus can be targeted by the immune system. In one aspect, the combined administration of immunostimulatory nucleic acids and cancer medicaments, particularly those which are classified as cancer immunotherapies, is useful for stimulating a specific immune response against a cancer antigen. A “cancer antigen” as used herein is a compound, such as a peptide, associated with a tumor or cancer cell surface and which is capable of provoking an immune response when expressed on the surface of an antigen presenting cell in the context of an MHC molecule. Cancer antigens, such as those present in cancer vaccines or those used to prepare cancer immunotherapies, can be prepared from crude cancer cell extracts, as described in Cohen, et al., 1994, Cancer Research, 54:1055, or by partially purifying the antigens, using recombinant technology, or de novo synthesis of known antigens. Cancer antigens can be used in the form of immunogenic portions of a particular antigen or in some instances a whole cell or a tumor mass can be used as the antigen. Such antigens can be isolated or prepared recombinantly or by any other means known in the art.

The theory of immune surveillance is that a prime function of the immune system is to detect and eliminate neoplastic cells before a tumor forms. A basic principle of this theory is that cancer cells are antigenically different from normal cells and thus elicit immune reactions that are similar to those that cause rejection of immunologically incompatible allografts. Studies have confirmed that tumor cells differ, either qualitatively or quantitatively, in their expression of antigens. For example, “tumor-specific antigens” are antigens that are specifically associated with tumor cells but not normal cells. Examples of tumor specific antigens are viral antigens in tumors induced by DNA or RNA viruses. “Tumor-associated” antigens are present in both tumor cells and normal cells but are present in a different quantity or a different form in tumor cells. Examples of such antigens are oncofetal antigens (e.g., carcinoembryonic antigen), differentiation antigens (e.g., T and Tn antigens), and oncogene products (e.g., HER/neu).

Different types of cells that can kill tumor targets in vitro and in vivo have been identified: natural killer cells (NK cells), cytolytic T lymphocytes (CTLs), lymphokine-activated killer cells (LAKs), and activated macrophages. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class I. CTLs are thought to be effector cells in the rejection of transplanted tumors and of tumors caused by DNA viruses. LAK cells are a subset of null lymphocytes distinct from the NK and CTL populations. Activated macrophages can kill tumor cells in a manner that is not antigen dependent nor MHC restricted once activated. Activated macrophages are thought to decrease the growth rate of the tumors they infiltrate. In vitro assays have identified other immune mechanisms such as antibody-dependent, cell-mediated cytotoxic reactions and lysis by antibody plus complement. However, these immune effector mechanisms are thought to be less important in vivo than the function of NK, CTLs, LAK, and macrophages in vivo (for review see Piessens, W. F., and David, J., “Tumor Immunology”, In: Scientific American Medicine, Vol. 2, Scientific American Books, N.Y., pp. 1-13, 1996.

The goal of immunotherapy is to augment a patient's immune response to an established tumor. One method of immunotherapy includes the use of adjuvants. Adjuvant substances derived from microorganisms, such as bacillus Calmette-Guerin, heighten the immune response and enhance resistance to tumors in animals.

Immunotherapeutic agents are medicaments which derive from antibodies or antibody fragments which specifically bind or recognize a cancer antigen. Antibody-based immunotherapies may function by binding to the cell surface of a cancer cell and thereby stimulate the endogenous immune system to attack the cancer cell.

As used herein a cancer antigen is broadly defined as an antigen expressed by a cancer cell. Preferably, the antigen is expressed at the cell surface of the cancer cell. Even more preferably, the antigen is one which is not expressed by normal cells, or at least not expressed to the same level as in cancer cells. For example, some cancer antigens are normally silent (i.e., not expressed) in normal cells, some are expressed only at certain stages of differentiation and others are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. The differential expression of cancer antigens in normal and cancer cells can be exploited in order to target cancer cells. As used herein, the terms “cancer antigen” and “tumor antigen” are used interchangeably.

Another way in which antibody-based therapy functions is as a delivery system for the specific targeting of toxic substances to cancer cells. Antibodies are usually conjugated to toxins such as ricin (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the cancer and non-specific toxicity to normal cells can be minimized.

In addition to the use of antibodies which are specific for cancer antigens, antibodies which bind to vasculature, such as those which bind to endothelial cells, are also useful in the invention. This is because generally solid tumors are dependent upon newly formed blood vessels to survive, and thus most tumors are capable of recruiting and stimulating the growth of new blood vessels. As a result, one strategy of many cancer medicaments is to attack the blood vessels feeding a tumor and/or the connective tissues (or stroma) supporting such blood vessels.

The use of immunostimulatory nucleic acids in conjunction with immunotherapeutic agents such as monoclonal antibodies is able to increase long-term survival through a number of mechanisms including significant enhancement of antibody-dependent cellular cytotoxicity, activation of natural killer (NK) cells and an increase in IFNα levels. The nucleic acids when used in combination with monoclonal antibodies serve to reduce the dose of the antibody required to achieve a biological result.

Examples of cancer immunotherapies which are currently being used or which are in development are listed in Table 4.

TABLE 4
Cancer Immunotherapies in Development or on the Market

| MARKETER | BRAND NAME (GENERIC NAME) | INDICATION |
|---|---|---|
| IDEC/Genentech, | Rituxan ™ (rituximab, Mabthera) (IDEC- | non-Hodgkin's lymphoma |
| Inc./Hoffmann-LaRoche (first | C2B8, chimeric murine/human anti-CD20 | |
| monoclonal antibody licensed for | MAb) | |
| the treatment of cancer in the | | |
| U.S.) | | |
| Genentech/Hoffmann-La Roche | Herceptin, anti-Her2 hMAb | Breast/ovarian |
| Cytogen Corp. | Quadramet (CYT-424) radiotherapeutic | Bone metastases |
| | agent | |
| Centocor/Glaxo/Ajinomoto | Panorex ® (17-1A) (murine monoclonal | Adjuvant therapy for |
| | antibody) | colorectal (Dukes-C) |
| Centocor/Ajinomoto | Panorex ® (17-1A) (chimeric murine | Pancreatic, lung, breast, |
| | monoclonal antibody) | ovary |
| IDEC | IDEC-Y2B8 (murine, anti-CD20 MAb | non-Hodgkin's lymhoma |
| | labeled with Yttrium-90) | |
| ImClone Systems | BEC2 (anti-idiotypic MAb, mimics the GD₃ | Small cell lung |
| | epitope) (with BCG) | |
| ImClone Systems | C225 (chimeric monoclonal antibody to | Renal cell |
| | epidermal growth factor receptor (EGFr)) | |
| Techniclone International/Alpha | Oncolym (Lym-1 monoclonal antibody | non-Hodgkin's lymphoma |
| Therapeutics | linked to 131 iodine) | |
| Protein Design Labs | SMART M195 Ab, humanized | Acute myleoid leukemia |
| Techniclone | ¹³¹I LYM-1 (Oncolym ™) | non-Hodgkin's lymphoma |
| Corporation/Cambridge Antibody | | |
| Technology | | |
| Aronex Pharmaceuticals, Inc. | ATRAGEN ® | Acute promyelocytic |
| | | leukemia |
| ImClone Systems | C225 (chimeric anti-EGFr monoclonal | Head & neck, non-small |
| | antibody) + cisplatin or radiation | cell lung cancer |
| Altarex, Canada | Ovarex (B43.13, anti-idiotypic CA125, | Ovarian |
| | mouse MAb) | |
| Coulter Pharma (Clinical results | Bexxar (anti-CD20 Mab labeled with ¹³¹I) | non-Hodgkin's lymphoma |
| have been positive, but the drug | | |
| has been associated with | | |
| significant bone marrow toxicity) | | |
| Aronex Pharmaceuticals, Inc. | ATRAGEN ® | Kaposi's sarcoma |
| IDEC Pharmaceuticals | Rituxan ™ (MAb against CD20) pan-B Ab in | B cell lymphoma |
| Corp./Genentech | combo. with chemotherapy | |
| LeukoSite/Ilex Oncology | LDP-03, huMAb to the leukocyte antigen | Chronic lymphocytic |
| | CAMPATH | leukemia (CLL) |
| Center of Molecular Immunology | ior t6 (anti CD6, murine MAb) CTCL | Cancer |
| Medarex/Novartis | MDX-210 (humanized anti-HER-2 bispecific | Breast, ovarian |
| | antibody) | |
| Medarex/Novartis | MDX-210 (humanized anti-HER-2 bispecific | Prostate, non-small cell |
| | antibody) | lung, pancreatic, breast |
| Medarex | MDX-11 (complement activating receptor | Acute myelogenous |
| | (CAR) monoclonal antibody) | leukemia (AML) |
| Medarex/Novartis | MDX-210 (humanized anti-HER-2 bispecific | Renal and colon |
| | antibody) | |
| Medarex | MDX-11 (complement activating receptor | Ex vivo bone marrow |
| | (CAR) monoclonal antibody) | purging in acute |
| | | myelogenous leukemia |
| | | (AML) |
| Medarex | MDX-22 (humanized bispecific antibody, | Acute myleoid leukemia |
| | MAb-conjugates) (complement cascade | |
| | activators) | |
| Cytogen | OV103 (Yttrium-90 labelled antibody) | Ovarian |
| Cytogen | OV103 (Yttrium-90 labelled antibody) | Prostate |
| Aronex Pharmaceuticals, Inc. | ATRAGEN ® | non-Hodgkin's lymphoma |
| Glaxo Wellcome plc | 3622W94 MAb that binds to EGP40 (17-1A) | non-small cell lung, |
| | pancarcinoma antigen on adenocarcinomas | prostate (adjuvant) |
| Genentech | Anti-VEGF, RhuMAb (inhibits | Lung, breast, prostate, |
| | angiogenesis) | colorectal |
| Protein Design Labs | Zenapax (SMART Anti-Tac (IL-2 receptor) | Leukemia, lymphoma |
| | Ab, humanized) | |
| Protein Design Labs | SMART M195 Ab, humanized | Acute promyelocytic |
| | | leukemia |
| ImClone Systems | C225 (chimeric anti-EGFr monoclonal | Breast |
| | antibody) + taxol | |
| ImClone Systems (licensed from | C225 (chimeric anti-EGFr monoclonal | prostate |
| RPR) | antibody) + doxorubicin | |
| ImClone Systems | C225 (chimeric anti-EGFr monoclonal | prostate |
| | antibody) + adriamycin | |
| ImClone Systems | BEC2 (anti-idiotypic MAb, mimics the GD₃ | Melanoma |
| | epitope) | |
| Medarex | MDX-210 (humanized anti-HER-2 bispecific | Cancer |
| | antibody) | |
| Medarex | MDX-220 (bispecific for tumors that express | Lung, colon, prostate, |
| | TAG-72) | ovarian, endometrial, |
| | | pancreatic and gastric |
| Medarex/Novartis | MDX-210 (humanized anti-HER-2 bispecific | Prostate |
| | antibody) | |
| Medarex/Merck KgaA | MDX-447 (humanized anti-EGF receptor | EGF receptor cancers |
| | bispecific antibody) | (head & neck, prostate, |
| | | lung, bladder, cervical, |
| | | ovarian) |
| Medarex/Novartis | MDX-210 (humanized anti-HER-2 bispecific | Comb. Therapy with G- |
| | antibody) | CSF for various cancers, |
| | | esp. breast |
| IDEC | MELIMMUNE-2 (murine monoclonal | Melanoma |
| | antibody therapeutic vaccine) | |
| IDEC | MELIMMUNE-1 (murine monoclonal | Melanoma |
| | antibody therapeutic vaccine) | |
| Immunomedics, Inc. | CEACIDE ™ (I-131) | Colorectal and other |
| NeoRx | Pretarget ™ radioactive antibodies | non-Hodgkin's B cell |
| | | lymphoma |
| Novopharm Biotech, Inc. | NovoMAb-G2 (pancarcinoma specific Ab) | Cancer |
| Techniclone Corporation/ | TNT (chimeric MAb to histone antigens) | Brain |
| Cambridge Antibody Technology | | |
| Techniclone International/ | TNT (chimeric MAb to histone antigens) | Brain |
| Cambridge Antibody Technology | | |
| Novopharm | Gliomab-H (Monoclonals - Humanized Abs) | Brain, melanomas, |
| | | neuroblastomas |
| Genetics Institute/AHP | GNI-250 Mab | Colorectal |
| Merck KgaA | EMD-72000 (chimeric-EGF antagonist) | Cancer |
| Immunomedics | LymphoCide (humanized LL2 antibody) | non-Hodgkin's B-cell |
| | | lymphoma |
| Immunex/AHP | CMA 676 (monoclonal antibody conjugate) | Acute myelogenous |
| | | leukemia |
| Novopharm Biotech, Inc. | Monopharm-C | Colon, lung, pancreatic |
| Novopharm Biotech, Inc. | 4B5 anti-idiotype Ab | Melanoma, small-cell |
| | | lung |
| Center of Molecular Immunology | ior egf/r3 (anti EGF-R humanized Ab) | Radioimmunotherapy |
| Center of Molecular Immunology | ior c5 (murine MAb colorectal) for | Colorectal |
| | radioimmunotherapy | |
| Creative BioMolecules/ | BABS (biosynthetic antibody binding site) | Breast cancer |
| Chiron | Proteins | |
| ImClone Systems/Chugai | FLK-2 (monoclonal antibody to fetal liver | Tumor-associated |
| | kinase-2 (FLK-2)) | angiogenesis |
| ImmunoGen, Inc. | Humanized MAb/small-drug conjugate | Small-cell lung |
| Medarex, Inc. | MDX-260 bispecific, targets GD-2 | Melanoma, glioma, |
| | | neuroblastoma |
| Procyon Biopharma, Inc. | ANA Ab | Cancer |
| Protein Design Labs | SMART 1D10 Ab | B-cell lymphoma |
| Protein Design Labs/Novartis | SMART ABL 364 Ab | Breast, lung, colon |
| Immunomedics, Inc. | ImmuRAIT-CEA | Colorectal |

Cancer vaccines are medicaments which are intended to stimulate an endogenous immune response against cancer cells. Currently produced vaccines predominantly activate the humoral immune system (i.e., the antibody dependent immune response). Other vaccines currently in development are focused on activating the cell-mediated immune system including cytotoxic T lymphocytes which are capable of killing tumor cells. Cancer vaccines generally enhance the presentation of cancer antigens to both antigen presenting cells (e.g., macrophages and dendritic cells) and/or to other immune cells such as T cells, B cells, and NK cells.

Although cancer vaccines may take one of several forms, as discussed infra, their purpose is to deliver cancer antigens and/or cancer associated antigens to antigen presenting cells (APC) in order to facilitate the endogenous processing of such antigens by APC and the ultimate presentation of antigen presentation on the cell surface in the context of MHC class I molecules. One form of cancer vaccine is a whole cell vaccine which is a preparation of cancer cells which have been removed from a subject, treated ex vivo and then reintroduced as whole cells in the subject. Lysates of tumor cells can also be used as cancer vaccines to elicit an immune response. Another form cancer vaccine is a peptide vaccine which uses cancer-specific or cancer-associated small proteins to activate T cells. Cancer-associated proteins are proteins which are not exclusively expressed by cancer cells (i.e., other normal cells may still express these antigens). However, the expression of cancer-associated antigens is generally consistently upregulated with cancers of a particular type. Yet another form of cancer vaccine is a dendritic cell vaccine which includes whole dendritic cells which have been exposed to a cancer antigen or a cancer-associated antigen in vitro. Lysates or membrane fractions of dendritic cells may also be used as cancer vaccines. Dendritic cell vaccines are able to activate antigen-presenting cells directly. Other cancer vaccines include ganglioside vaccines, heat-shock protein vaccines, viral and bacterial vaccines, and nucleic acid vaccines.

In some embodiments, it is envisioned that immunostimulatory nucleic acids can be used in the manufacture of cancer vaccines, particularly dendritic cell based vaccines. As an example, a population of cancer cells, such as prostate cancer cells, may be exposed to an immunostimulatory nucleic acid, such as a poly-G nucleic acid, after which they are exposed to a dendritic cell population. The poly-G nucleic acid can stimulate apoptosis of the cancer cells thereby facilitating antigen processing by the dendritic cells. Alternatively, the immunostimulatory nucleic acid may be included in a cancer vaccine in order to prime dendritic cells prior to or at the time of their contact with cancer cells and/or antigens.

The use of immunostimulatory nucleic acids in conjunction with cancer vaccines provides an improved antigen-specific humoral and cell mediated immune response, in addition to activating NK cells and endogenous dendritic cells, and increasing IFNoc levels. This enhancement allows a vaccine with a reduced antigen dose to be used to achieve the same beneficial effect.

In some instances, cancer vaccines may be used along with adjuvants. Adjuvants are substances which activate the subject's immune system, and can be used as an adjunct therapy in any of the methods of the invention. Adjuvants include Alum, QS-Stimulon (Aquila), MF-59 (Chiron), Detox (Ribi), Optivax (Vaxcels) and LeIF (Corixa).

Other vaccines take the form of dendritic cells which have been exposed to cancer antigens in vitro, have processed the antigens and are able to express the cancer antigens at their cell surface in the context of MHC molecules for effective antigen presentation to other immune system cells.

The immunostimulatory nucleic acids are used in one aspect of the invention in conjunction with cancer vaccines which are dendritic cell based. A dendritic cell is a professional antigen presenting cell. Dendritic cells form the link between the innate and the acquired immune system by presenting antigens and through their expression of pattern recognition receptors which detect microbial molecules like LPS in their local environment. Dendritic cells efficiently internalize, process, and present soluble specific antigen to which it is exposed. The process of internalizing and presenting antigen causes rapid upregulation of the expression of major histocompatibility complex (MHC) and costimulatory molecules, the production of cytokines, and migration toward lymphatic organs where they are believed to be involved in the activation of T cells.

Table 5 lists a variety of cancer vaccines which are either currently being used or are in development.

TABLE 5
Cancer Vaccines in Development or on the Market

| MARKETER | BRAND NAME (GENERIC NAME) | INDICATION |
|---|---|---|
| Center of Molecular | EGF | Cancer |
| Immunology | | |
| Center of Molecular | | Ganglioside cancer |
| Immunology | | vaccine |
| Center of Molecular | Anti-idiotypic | Cancer vaccine |
| Immunology | | |
| ImClone Systems/Memorial | Gp75 antigen | Melanoma |
| Sloan-Kettering Cancer Center | | |
| ImClone Systems/Memorial | Anti-idiotypic Abs | Cancer vaccines |
| Sloan-Kettering Cancer Center | | |
| Progenics Pharmaceuticals, Inc. | GMK melanoma vaccine | Melanoma |
| Progenics Pharmaceuticals, Inc, | MGV ganglioside conjugate vaccine | Lymphoma, |
| | | colorectal, lung |
| Corixa | Her2/neu | Breast, ovarian |
| AltaRex | Ovarex | Ovarian |
| AVAX Technologies Inc. | M-Vax, autologous whole cell | Melanoma |
| AVAX Technologies Inc. | O-Vax, autologous whole cell | Ovarian |
| AVAX Technologies Inc. | L-Vax, autologous whole cell | Leukemia-AML |
| Biomira Inc./Chiron | Theratope, STn-KLH | Breast, Colorectal |
| Biomira Inc. | BLP25, MUC-1 peptide vaccine encapsulated | Lung |
| | in liposomal delivery system | |
| Biomira Inc. | BLP25, MUC-1 peptide vaccine encapsulated | Lung |
| | in liposomal delivery system + Liposomal IL-2 | |
| Biomira Inc. | Liposomal idiotypic vaccine | Lymphoma B-cell |
| | | malignancies |
| Ribi Immunochem | Melacine, cell lysate | Melanoma |
| Corixa | Peptide antigens, microsphere delivery sysem | Breast |
| | and LeIF adjuvant | |
| Corixa | Peptide antigens, microsphere delivery sysem | Prostate |
| | and LeIF adjuvant | |
| Corixa | Peptide antigens, microsphere delivery sysem | Ovarian |
| | and LeIF adjuvant | |
| Corixa | Peptide antigens, microsphere delivery sysem | Lymphoma |
| | and LeIF adjuvant | |
| Corixa | Peptide antigens, microsphere delivery sysem | Lung |
| | and LeIF adjuvant | |
| Virus Research Institute | Toxin/antigen recombinant delivery system | All cancers |
| Apollon Inc. | Genevax-TCR | T-cell lymphoma |
| Bavarian Nordic Research | MVA-based (vaccinia virus) vaccine | Melanoma |
| Institute A/S | | |
| BioChem Pharma/BioChem | PACIS, BCG vaccine | Bladder |
| Vaccine | | |
| Cantab Pharmaceuticals | TA-HPV | Cervical |
| Cantab Pharmaceuticals | TA-CIN | Cervical |
| Cantab Pharmaceuticals | DISC-Virus, immunotherapy | Cancer |
| Pasteur Merieux Connaught | ImmuCyst ®/TheraCys ® - BCG | Bladder |
| | Immunotherapeutic (Bacillus Calmette- | |
| | Guerin/Connaught), for intravesical treatment | |
| | of superficial bladder cancer | |

As used herein, chemotherapeutic agents are chemical and biological agents which target cancer cells directly. Some of these agents function to inhibit a cellular activity which the cancer cell is dependent upon for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. Combination chemotherapy and immunostimulatory nucleic acid administration increases the maximum tolerable dose of chemotherapy.

Examples of chemotherapeutic agents which can be used according to the invention include but are not limited to Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCI, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

Chemotherapeutic agents which are currently in development or in use in a clinical setting are shown in Table 6.

TABLE 6
Cancer Drugs in Development or on the Market

| Marketer | Brand Name | Generic Name | Indication |
|---|---|---|---|
| Abbott | TNP 470/AGM 1470 | Fragyline | Anti-Angiogenesis in Cancer |
| Takeda | TNP 470/AGM 1470 | Fragyline | Anti-Angiogenesis in Cancer |
| Scotia | Meglamine GLA | Meglamine GLA | Bladder Cancer |
| Medeva | Valstar | Valrubicin | Bladder Cancer - Refractory |
| | | | in situ carcinoma |
| Medeva | Valstar | Valrubicin | Bladder Cancer - Papillary |
| | | | Cancer |
| Rhone Poulenc | Gliadel Wafer | Carmustaine + Polifepr Osan | Brain Tumor |
| Warner Lambert | Undisclosed Cancer (b) | Undisclosed Cancer (b) | Cancer |
| Bristol Myers | RAS Famesyl Transferase | RAS FamesylTransferase | Cancer |
| Squib | Inhibitor | Inhibitor | |
| Novartis | MMI 270 | MMI 270 | Cancer |
| Bayer | BAY 12-9566 | BAY 12-9566 | Cancer |
| Merck | Famesyl Transferase Inhibitor | Famesyl Transferase | Cancer (Solid tumors - |
| | | Inhibitor | pancrease, colon, lung, |
| | | | breast) |
| Pfizer | PFE | MMP | Cancer, angiogenesis |
| Pfizer | PFE | Tyrosine Kinase | Cancer, angiogenesis |
| Lilly | MTA/LY 231514 | MTA/LY 231514 | Cancer Solid Tumors |
| Lilly | LY 264618/Lometexol | Lometexol | Cancer Solid Tumors |
| Scotia | Glamolec | LiGLA (lithium-gamma | Cancer, pancreatic, breast, |
| | | linolenate) | colon |
| Warner Lambert | CI-994 | CI-994 | Cancer, Solid Tumors/ |
| | | | Leukemia |
| Schering AG | Angiogenesis inhibitor | Angiogenesis Inhibitor | Cancer/Cardio |
| Takeda | TNP-470 | n/k | Malignant Tumor |
| Smithkline | Hycamtin | Topotecan | Metastatic Ovarian Cancer |
| Beecham | | | |
| Novartis | PKC 412 | PKC 412 | Multi-Drug Resistant Cancer |
| Novartis | Valspodar | PSC 833 | Myeloid Leukemia/Ovarian |
| | | | Cancer |
| Immunex | Novantrone | Mitoxantrone | Pain related to hormone |
| | | | refractory prostate cancer. |
| Warner Lambert | Metaret | Suramin | Prostate |
| Genentech | Anti-VEGF | Anti-VEGF | Prostate/Breast/Colorectal/ |
| | | | NSCL Cancer |
| British Biotech | Batimastat | Batimastat (BB94) | Pterygium |
| Eisai | E 7070 | E 7070 | Solid Tumors |
| Biochem | BCH-4556 | BCH-4556 | Solid Tumors |
| Pharma | | | |
| Sankyo | CS-682 | CS-682 | Solid Tumors |
| Agouron | AG2037 | AG2037 | Solid Tumors |
| IDEC Pharma | 9-AC | 9-AC | Solid Tumors |
| Agouron | VEGF/b-FGF Inhibitors | VEGF/b-FGF Inhibitors | Solid Tumors |
| Agouron | AG3340 | AG3340 | Solid Tumors/Macular |
| | | | Degen |
| Vertex | Incel | VX-710 | Solid Tumors - IV |
| Vertex | VX-853 | VX-853 | Solid Tumors - Oral |
| Zeneca | ZD 0101 (inj) | ZD 0101 | Solid Tumors |
| Novartis | ISI 641 | ISI 641 | Solid Tumors |
| Novartis | ODN 698 | ODN 698 | Solid Tumors |
| Tanube Seiyaku | TA 2516 | Marimistat | Solid Tumors |
| British Biotech | Marimastat | Marimastat (BB 2516) | Solid Tumors |
| Celltech | CDP 845 | Aggrecanase Inhibitor | Solid Tumors/Breast |
| | | | Cancer |
| Chiroscience | D2163 | D2163 | Solid Tumors/Metastases |
| Warner Lambert | PD 183805 | PD 183805 | |
| Daiichi | DX8951f | DX8951f | Anti-Cancer |
| Daiichi | Lemonal DP 2202 | Lemonal DP 2202 | Anti-Cancer |
| Fujisawa | FK 317 | FK 317 | Anticancer Antibiotic |
| Chugai | Picibanil | OK-432 | Antimalignant Tumor |
| Nycomed | AD 32/valrubicin | Valrubicin | Bladder Cancer-Refractory |
| Amersham | | | Insitu Carcinoma |
| Nycomed | Metastron | Strontium Derivative | Bone Cancer (adjunt therapy, |
| Amersham | | | Pain) |
| Schering Plough | Temodal | Temozolomide | Brain Tumours |
| Schering Plough | Temodal | Temozolonide | Brain Tumours |
| Liposome | Evacet | Doxorubicin, Liposomal | Breast Cancer |
| Nycomed | Yewtaxan | Paclitaxel | Breast Cancer Advanced, |
| Amersham | | | Ovarian Cancer Advanced |
| Bristol Myers | Taxol | Paclitaxel | Breast Cancer Advanced, |
| Squib | | | Ovarian Cancer Advanced, |
| | | | NSCLC |
| Roche | Xeloda | Capecitabine | Breast Cancer, Colorectal |
| | | | Cancer |
| Roche | Furtulon | Doxifluridine | Breast Cancer, Colorectal |
| | | | Cancer, Gastric Cancer |
| Pharmacia & | Adriamycin | Doxorubicin | Breast Cancer, Leukemia |
| Upjohn | | | |
| Ivax | Cyclopax | Paclitaxel, Oral | Breast/Ovarian Cancer |
| Rhone Poulenc | Oral Taxoid | Oral Taxoid | Broad Cancer |
| AHP | Novantrone | Mitoxantrone | Cancer |
| Sequus | SPI-077 | Cisplatin, Stealth | Cancer |
| Hoechst | HMR 1275 | Flavopiridol | Cancer |
| Pfizer | CP-358, 774 | EGFR | Cancer |
| Pfizer | CP-609, 754 | RAS Oncogene Inhibitor | Cancer |
| Bristol Myers | BMS-182751 | Oral Platinum | Cancer (Lung, Ovarian) |
| Squib | | | |
| Bristol Myers | UFT (Tegafur/Uracil) | UFT (Tegafur/Uracil) | Cancer Oral |
| Squib | | | |
| Johnson & | Ergamisol | Levamisole | Cancer Therapy |
| Johnson | | | |
| Glaxo Wellcome | Eniluracil/776C85 | 5FU Enhancer | Cancer, Refractory Solid & |
| | | | Colorectal Cancer |
| Johnson & | Ergamisol | Levamisole | Colon Cancer |
| Johnson | | | |
| Rhone Poulenc | Campto | Irinotecan | Colorectal Cancer, Cervical |
| | | | Cancer |
| Pharmacia & | Camptosar | Irinotecan | Colorectal Cancer, Cervical |
| Upjohn | | | Cancer |
| Zeneca | Tomudex | Ralitrexed | Colorectal Cancer, Lung |
| | | | Cancer, Breast Cancer |
| Johnson & | Leustain | Cladribine | Hairy Cell Leukaemia |
| Johnson | | | |
| Ivax | Paxene | Paclitaxel | Kaposi Sarcoma |
| Sequus | Doxil | Doxorubicin, Liposomal | KS/Cancer |
| Sequus | Caelyx | Doxorubicin, Liposomal | KS/Cancer |
| Schering AG | Fludara | Fludarabine | Leukaemia |
| Pharmacia & | Pharmorubicin | Epirubicin | Lung/Breast Cancer |
| Upjohn | | | |
| Chiron | DepoCyt | DepoCyt | Neoplastic Meningitis |
| Zeneca | ZD1839 | ZD 1839 | Non Small Cell Lung |
| | | | Cancer, Pancreatic Cancer |
| BASF | LU 79553 | Bis-Naphtalimide | Oncology |
| BASF | LU 103793 | Dolastain | Oncology |
| Shering Plough | Caetyx | Doxorubicin-Liposome | Ovarian/Breast Cancer |
| Lilly | Gemzar | Gemcitabine | Pancreatic Cancer, Non |
| | | | Small Cell Lung Cancer, |
| | | | Breast, Bladder and Ovarian |
| Zeneca | ZD 0473/Anormed | ZD 0473/Anormed | Platinum based NSCL, |
| | | | ovarian etc. |
| Yamanouchi | YM 116 | YM 116 | Prostate Cancer |
| Nycomed | Seeds/I-125 Rapid St | Lodine Seeds | Prostate Cancer |
| Amersham | | | |
| Agouron | Cdk4/cdk2 inhibitors | cdk4/cdk2 inhibitors | Solid Tumors |
| Agouron | PARP inhibitors | PARP Inhibitors | Solid Tumors |
| Chiroscience | D4809 | Dexifosamide | Solid Tumors |
| Bristol Myers | UFT (Tegafur/Uracil) | UFT (Tegafur/Uracil) | Solid Tumors |
| Squib | | | |
| Sankyo | Krestin | Krestin | Solid Tumors |
| Asta Medica | Ifex/Mesnex | Ifosamide | Solid Tumors |
| Bristol Meyers | Ifex/Mesnex | Ifosamide | Solid Tumors |
| Squib | | | |
| Bristol Myers | Vumon | Teniposide | Solid Tumors |
| Squib | | | |
| Bristol Myers | Paraplatin | Carboplatin | Solid Tumors |
| Squib | | | |
| Bristol Myers | Plantinol | Cisplatin, Stealth | Solid Tumors |
| Squib | | | |
| Bristol Myers | Plantinol | Cisplatin | Solid Tumors |
| Squib | | | |
| Bristol Myers | Vepeside | Etoposide | Solid Tumors Melanoma |
| Squib | | | |
| Zeneca | ZD 9331 | ZD 9331 | Solid Tumors, Advanced |
| | | | Colorectal |
| Chugai | Taxotere | Docetaxel | Solid Tumors, Breast Cancer |
| Rhone Poulenc | Taxotere | Docetaxel | Solid Tumors, Breast Cancer |
| Glaxo Wellcome | Prodrug of guanine | prodrug of arabinside | T Cell Leukemia/Lymphoma |
| | arabinside | | & B Cell Neoplasm |
| Bristol Myers | Taxane Analog | Taxane Analog | Taxol follow up |
| Squib | | | |

Hormone therapy refers to the use of hormones or hormone substitutes and derivatives in the treatment of subjects having or at risk of having cancer. Examples include estrogen therapy e.g., diethylstilbestrol and ethinyl estradiol (e.g., for breast cancer and prostate cancer), anti-estrogen therapy e.g., tamoxifen (e.g., for breast cancer), progestin therapy e.g., medroxyprogesterone and megestrol acetate (e.g., for breast cancer and endometrial cancer), androgen blockade e.g., anti-androgens such as flutamide (e.g., for prostate cancer), adrenocorticosteroids including adrenal steroids (e.g., for lymphocytic leukemias and lymphomas), synthetic glucocorticoid therapy e.g., prednisone, methylprednisone, and dexamethasone (e.g., for breast cancer, and some CNS neoplasias), androgens e.g., fluoxymesterone (e.g., for breast cancer), synthetic testosterone analogs, aromatase inhibitor e.g., aminoglutethimide (e.g., for breast cancer), gonadotropin-releasing hormone agonists e.g., leuprolide (e.g., for prostate cancer), somatostatin analogs e.g., octreotide (e.g., for gastric cancer and pancreatic cancers). In important embodiments, the combination of immunostimulatory nucleic acids and hormone therapy is used in breast cancer and prostate cancer.

Biological response modifiers are agents that alter a subject's response to cancer rather than by direct cytotoxicity of the cancer cells. Examples include cytokines e.g., type I interferons (α and β), type II interferon (γ), interleukins (e.g., IL-2, IL-1α and IL-1β), and TNFα and TNF-β; and hemopoietic growth factors e.g., erythropoietin, GM-CSF, and G-CSF.

In one embodiment, the methods of the invention use immunostimulatory nucleic acids as a replacement to the use of IFNα therapy in the treatment of cancer. Currently, some treatment protocols call for the use of IFNα. Since IFNα is produced following the administration of some immunostimulatory nucleic acids, these nucleic acids can be used to generate IFNα endogenously.

In yet other embodiments, the immunostimulatory nucleic acids and the cancer medicaments of the invention may be administered along with IFNα (e.g., Intron A). In these latter embodiments, subjects would receive an immunostimulatory nucleic acid of the invention such as, for example, a CpG nucleic acid, a poly-G nucleic acid, or a nucleic acid with a phosphorothioate modified backbone, as well as a cancer medicament such as one or more chemotherapeutic agents, immunotherapeutic agents, cancer vaccines, biological response modifiers and hormone therapies, and interferon-α. The immunostimulatory nucleic acid may also be a nucleic acid which is free of a CpG motif, a T-rich motif and a poly-G motif. In some important embodiments involving the administration of interferon-α (e.g., Intron A, Schering Plough), the immunostimulatory nucleic acid is not a CpG nucleic acid.

The term “effective amount” of a immunostimulatory nucleic acid refers to the amount necessary or sufficient to realize a desired biologic effect. For example, an effective amount of an immunostimulatory nucleic acid could be that amount necessary to cause activation of the immune system, resulting potentially in the development of an antigen specific immune response. According to some aspects of the invention, an effective amount is that amount of an immunostimulatory nucleic acid and that amount of a cancer medicament, which when combined or co-administered, results in a synergistic response to the cancer, either in the prevention or the treatment of the cancer. A synergistic amount is that amount which produces an anti-cancer response that is greater than the sum of the individual effects of either the immunostimulatory nucleic acid and the cancer medicament alone. For example, a synergistic combination of an immunostimulatory nucleic acid and a cancer medicament provides a biological effect which is greater than the combined biological effect which could have been achieved using each of the components (i.e., the nucleic acid and the medicament) separately. The biological effect may be the amelioration and or absolute elimination of symptoms resulting from the cancer. In another embodiment, the biological effect is the complete abrogation of the cancer, as evidenced for example, by the absence of a tumor or a biopsy or blood smear which is free of cancer cells.

The effective amount of immunostimulatory nucleic acid necessary to synergize with a cancer medicament in the treatment of a cancer or in the reduction of the risk of developing a cancer may vary depending upon the sequence of the immunostimulatory nucleic acid, the backbone constituents of the nucleic acid, and the mode of delivery of the nucleic acid. The effective amount for any particular application can also vary depending on such factors as the cancer being treated, the particular immunostimulatory nucleic acid being administered (e.g. the nature, number or location of immunostimulatory motifs in the nucleic acid), the size of the subject, or the severity of the disease or condition. One of ordinary skill in the art can empirically determine the effective amount of a particular immunostimulatory nucleic acid and cancer medicament combination without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

In some embodiments, the immunostimulatory nucleic acids are administered in an effective amount to stimulate or induce a Th1 immune response, or a Th2 immune response, or a general immune response. An effective amount to stimulate a Th1 immune response may be defined as that amount which stimulates the production of one or more Th1-type cytokines such as interleukin 2 (IL-2), IL-12, tumor necrosis factor (TNFα) and interferon gamma (IFN-γ), and/or production of one or more Th1-type antibodies. An effective amount to stimulate a Th2 immune response, on the other hand, may be defined as that amount which stimulates the production of one or more Th2-type cytokines such as IL-4, IL-5, IL-6, IL-9, IL-10 and IL-13, and/or the production of one or more Th2-type antibodies.

In some embodiments of the invention, the immunostimulatory nucleic acid is administered in an effective amount for preventing bacterial, viral or fungal infection. Immunostimulatory nucleic acids are known to be useful for preventing bacterial and viral infections. Bacterial, viral and fungal infections present a challenge to the immunocompromised cancer patient, and much cancer patient management is focused on preventing such infections, particularly since cancer patients are less likely to mount an effective immune response. In one embodiment, the cancer medicament is first administered to the subject when the cancer is diagnosed and the immunostimulatory nucleic acid is administered to the subject in an amount effective to prevent bacterial, viral or fungal infection after the administration of the cancer medicament and potentially when the subject exhibits signs of neutropenia. In another embodiment, the cancer medicament and the immunostimulatory nucleic acid are administered at the same time.

In some instances, a sub-therapeutic dosage of either the immunostimulatory nucleic acid or the cancer medicament, or a sub-therapeutic dosage of both, is used in the treatment of a subject having, or at risk of developing, cancer. As an example, it has been discovered according to the invention, that when the two classes of drugs are used together, the cancer medicament can be administered in a sub-therapeutic dose and still produce a desirable therapeutic result. A “sub-therapeutic dose” as used herein refers to a dosage which is less than that dosage which would produce a therapeutic result in the subject if administered in the absence of the other agent. Thus, the sub-therapeutic dose of a cancer medicament is one which would not produce the desired therapeutic result in the subject in the absence of the administration of the immunostimulatory nucleic acid. Therapeutic doses of cancer medicaments are well known in the field of medicine for the treatment of cancer. These dosages have been extensively described in references such as Remington's Pharmaceutical Sciences, 18th ed., 1990; as well as many other medical references relied upon by the medical profession as guidance for the treatment of cancer. Therapeutic dosages of immunostimulatory nucleic acids have also been described in the art and methods for identifying therapeutic dosages in subjects are described in more detail herein.

In other aspects, the method of the invention involves administering a dose of a cancer medicament to a subject, without inducing side effects, due to the administration of an immunostimulatory nucleic acid. Ordinarily, when a cancer medicament is administered to a subject in a therapeutic dose, a variety of side effects can occur. The severity of these side effects, in some instances, increase with increasing dosage of the cancer medicament. It is for this reason that cancer medicaments are usually administered at the lowest possible therapeutic dose in order to prevent the occurrence of the adverse side effects. (Discussed in more detail above, as well as in the medical literature). Consequently, cancer medicaments are not ordinarily administered in high therapeutic doses, no matter what therapeutic benefits are derived. However, it was discovered, according to the invention, that high doses of cancer medicaments which ordinarily induce side effects can be administered without inducing the side effects as long as the subject also receives an immunostimulatory nucleic acid. The type and extent of the side effects ordinarily induced by the cancer medicament will depend on the particular cancer medicament used. Thus the invention provides methods for reducing side effects resulting from the administration of low or high therapeutic doses of cancer medicaments.

Some aspects of the invention call for the administration of an immunostimulatory nucleic acid in an effective amount to inhibit the induction of side effects by a cancer medicament when the cancer medicament is administered in a dose which ordinarily, if administered by itself, would induce side effects. An effective amount of an immunostimulatory nucleic acid to inhibit the induction of side effects may be defined as the effective amount to inhibit a microbial (e.g., bacterial, fungal, parasitic and viral) infection. The effective amount to inhibit the induction of side effects may also be that amount which inhibits myelosuppression in the form of anemia, neutropenia and thrombocytopenia. Yet another measure of the effective amount to inhibit the induction of side effects is that amount which inhibits an adverse allergic reaction, such as that which is sometimes experienced during a blood product transfusion, or in response to certain medications.

For any compound described herein a therapeutically effective amount can be initially determined from cell culture assays. In particular, the effective amount of immunostimulatory nucleic acid can be determined using in vitro stimulation assays. The stimulation index of the immunostimulatory nucleic acid can be compared to that of previously tested immunostimulatory acids. The stimulation index can be used to determine an effective amount of the particular oligonucleotide for the particular subject, and the dosage can be adjusted upwards or downwards to achieve the desired levels in the subject.

Therapeutically effective amounts can also be determined in animal studies. For instance, the effective amount of immunostimulatory nucleic acid and cancer medicament to induce a synergistic response can be assessed using in vivo assays of tumor regression and/or prevention of tumor formation. Relevant animal models include assays in which malignant cells are injected into the animal subjects, usually in a defined site. Generally, a range of immunostimulatory nucleic acid doses are administered into the animal along with a range of cancer medicament doses. Inhibition of the growth of a tumor following the injection of the malignant cells is indicative of the ability to reduce the risk of developing a cancer. Inhibition of further growth (or reduction in size) of a pre-existing tumor is indicative of the ability to treat the cancer. Mice which have been modified to have human immune system elements can be used as recipients of human cancer cell lines to determine the effective amount of the synergistic combination.

A therapeutically effective dose can also be determined from human data for immunostimulatory nucleic acids which have been tested in humans (human clinical trials have been initiated) and for compounds which are known to exhibit similar pharmacological activities, such as other adjuvants, e.g., LT and other antigens for vaccination purposes.

The applied dose of both the immunostimulatory nucleic acid and the cancer medicament can be adjusted based on the relative bioavailability and potency of the administered compounds, including the adjuvants used. Adjusting the dose to achieve maximal efficacy based on the methods described above and other methods are well within the capabilities of the ordinarily skilled artisan. Most of the cancer medicaments have been identified. These amounts can be adjusted when they are combined with immuno-stimulatory nucleic acids by routine experimentation.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

In other embodiments of the invention, the immunostimulatory nucleic acid is administered on a routine schedule. The cancer medicament may also be administered on a routine schedule, but alternatively, may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the immunostimulatory nucleic acid on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the immunostimulatory nucleic acid on a daily basis for the first week, followed by a monthly basis for several months, and then every three months after that. Any particular combination would be covered by the routine schedule as long as it is determined ahead of time that the appropriate schedule involves administration on a certain day.

In methods directed at subjects at risk of developing a cancer (e.g., either by known or expected exposure to a carcinogen or through a genetic or familial predisposition to cancer), timing of the administration of the immunostimulatory nucleic acid and the cancer medicament may also be particularly important. For instance, in a subject with a genetic predisposition to cancer, the immunostimulatory nucleic acid and the cancer medicament, preferably in the form of an immunotherapy or a cancer medicament, may be administered to the subject on a regular basis. Additionally the immunostimulatory nucleic acid and the cancer medicament, again preferably in the form of an immunotherapy or a cancer vaccine, may be administered to persons who will likely be exposed to a carcinogen.

The methods and compositions of the invention aim to treat subjects having or at risk of developing a cancer. As used herein, the treatment of such subjects therefore embraces treatment prior to and after the existence of a cancer. Treatment after a cancer has started aims to reduce, ameliorate or altogether eliminate the cancer, and/or its associated symptoms, or prevent it from becoming worse. Treatment of subjects before a cancer has started (i.e., prophylactic treatment) aims to reduce the risk of developing the cancer. As used herein, the term “prevent” refers to the prophylactic treatment of cancer in patients who are at risk of developing a cancer (resulting in a decrease in the probability that the subject will develop a cancer), and to the inhibition of further growth of an already established cancer.

The immunostimulatory nucleic acids may be delivered to the subject in the form of a plasmid vector. In some embodiments, one plasmid vector could include both the immunostimulatory nucleic acid and a nucleic acid encoding a cancer medicament, if the cancer medicament can be encoded by a nucleic acid. In other embodiments, separate plasmids could be used. In yet other embodiments, no plasmids could be used.

The compositions of the invention may be delivered to the cancer (e.g., to a tumor) or to the immune system or both. In its broadest sense, a “vector” is any vehicle capable of facilitating the transfer of the compositions to the target cells. The vector generally transports the nucleic acid and/or the cancer medicament to the target cells with reduced degradation relative to the extent of degradation that would result in the absence of the vector.

In general, the vectors useful in the invention are divided into two classes: biological vectors and chemical/physical vectors. Biological vectors and chemical/physical vectors are useful in the delivery and/or uptake of nucleic acids and cancer medicaments.

Biological vectors include, but are not limited to, plasmids, phagemids, viruses, other vehicles derived from viral or bacterial sources that have been manipulated by the insertion or incorporation of nucleic acid sequences, and free nucleic acid fragments which can be attached to nucleic acid sequences. Viral vectors are a preferred type of biological vector and include, but are not limited to, nucleic acid sequences from the following viruses: retroviruses, such as: Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One can readily employ other viral vectors not named but known in the art.

Preferred viral vectors are based on non-cytopathic eukaryotic viruses in which non-essential genes have been replaced with a nucleic acid of interest. Non-cytopathic viruses include retroviruses, the life cycle of which involves reverse transcription of genomic viral RNA into DNA with subsequent proviral integration into host cellular DNA. Retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Cliffton, N.J. (1991).

Another preferred virus for certain applications is the adeno-associated virus, a double-stranded DNA virus. The adeno-associated virus can be engineered to be replication-deficient and is capable of infecting a wide range of cell types and species. It further has advantages, such as heat and lipid solvent stability; high transduction frequencies in cells of diverse lineages; and lack of superinfection inhibition thus allowing multiple series of transductions. Reportedly, the adeno-associated virus can integrate into human insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other biological vectors include plasmid vectors. Plasmid vectors have been extensively described in the art and are well-known to those of skill in the art. See e.g., Sambrook et al., “Molecular Cloning: A Laboratory Manual,” Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom designed using restriction enzymes and ligation reactions to remove and add specific fragments of DNA.

In addition to the biological vectors, chemical/physical vectors may be used to deliver a nucleic acid and/or a cancer medicament to a target cell and facilitate uptake thereby. As used herein, a “chemical/physical vector” refers to a natural or synthetic molecule, other than those derived from bacteriological or viral sources, capable of delivering the nucleic acid and/or a cancer medicament.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA and intact virions can be encapsulated within the aqueous interior and be delivered to cells in a biologically active form (Fraley, et al., Trends Biochem. Sci., (1981) 6:77).

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”. PCT/US/0307 describes a biocompatible, preferably biodegradable polymeric matrix for containing an exogenous gene under the control of an appropriate promoter. The polymeric matrix can be used to achieve sustained release of the immunostimulatory nucleic acid and/or the cancer medicament in the subject.

The polymeric matrix preferably is in the form of a microparticle such as a microsphere (wherein the nucleic acid and/or the cancer medicament is dispersed throughout a solid polymeric matrix) or a microcapsule (wherein the nucleic acid and/or cancer medicament is stored in the core of a polymeric shell). Other forms of the polymeric matrix for containing the nucleic acid and/or the cancer medicament include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the cancer medicament are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the immunostimulatory nucleic acids are administered to the subject via an implant while the cancer medicament is administered acutely.

The mode of delivery of the cancer medicament is dependent upon the nature of the medicament, the specificity of the medicament for the cancer, and its inherent stability in vivo. As an example, chemotherapeutic agents which target dividing cells are more preferably administered locally or systemically for a short period of time (e.g., in an intravenous bolus). In contrast, immunotherapeutic agents or cancer vaccines which are more selective for the particular cancer to be treated (as compared to a chemotherapeutic agent) may be more suitable for sustained release formulations.

In another embodiment the chemical/physical vector is a biocompatible microsphere that is suitable for delivery, such as oral or mucosal delivery. Such microspheres are disclosed in Chickering et al., Biotech. And Bioeng., (1996) 52:96-101 and Mathiowitz et al., Nature, (1997) 386:.410-414 and PCT Patent Application WO97/03702.

Both non-biodegradable and biodegradable polymeric matrices can be used to deliver the nucleic acid and/or the cancer medicament to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the immunostimulatory nucleic acids. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake by a target cell of the nucleic acid and/or the cancer medicament include calcium phosphate and other chemical mediators of intracellular transport, microinjection compositions, electroporation and homologous recombination compositions (e.g., for integrating a nucleic acid into a preselected location within the target cell chromosome).

The immunostimulatory nucleic acid and the cancer medicament may be administered alone (e.g. in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The immunostimulatory nucleic acid and cancer medicament can be combined with other therapeutic agents such as adjuvants to enhance immune responses even further. The immunostimulatory nucleic acid, cancer medicament and other therapeutic agent may be administered simultaneously or sequentially. When the other therapeutic agents are administered simultaneously they can be administered in the same or separate formulations, but are administered at the same time. The administration of the other therapeutic agents (such as adjuvants) and the immunostimulatory nucleic acid and cancer medicament can also be temporally separated, meaning that the therapeutic agents are administered at a different time, either before or after, the administration of the immunostimulatory nucleic acid and the cancer medicament. The separation in time between the administration of these compounds may be a matter of minutes or it may be longer. Other therapeutic agents include but are not limited to non-nucleic acid adjuvants, cytokines, non-immunotherapeutic antibodies, antigens, etc.

A “non-nucleic acid adjuvant” is any molecule or compound except for the immunostimulatory nucleic acids described herein which can stimulate the humoral and/or cellular immune response. Non-nucleic acid adjuvants include, for instance, adjuvants that create a depo effect, immune stimulating adjuvants, adjuvants that create a depo effect and stimulate the immune system and mucosal adjuvants.

An “adjuvant that creates a depo effect” as used herein is an adjuvant that causes an antigen, such as a cancer antigen present in a cancer vaccine, to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

An “immune stimulating adjuvant” is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

“Adjuvants that create a depo effect and stimulate the immune system” are those compounds which have both of the above-identified functions. This class of adjuvants includes but is not limited to ISCOMS (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A “non-nucleic acid mucosal adjuvant” as used herein is an adjuvant other than an immunostimulatory nucleic acid that is capable of inducing a mucosal immune response in a subject when administered to a mucosal surface in conjunction with an antigen. Mucosal adjuvants include but are not limited to Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); andCTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis)(Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worster, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Immune responses can also be induced or augmented by the co-administration or co-linear expression of cytokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997) with the immunostimulatory nucleic acids and cancer medicaments. The cytokines can be administered directly with immunostimulatory nucleic acids or may be administered in the form of a nucleic acid vector that encodes the cytokine, such that the cytokine can be expressed in vivo. In one embodiment, the cytokine is administered in the form of a plasmid expression vector. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Cytokines also are central in directing the T cell response. Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand. In some embodiments, the cytokine is a Th1 cytokine. In still other embodiments, the cytokine is a Th2 cytokine.

In other aspects, the invention relates to kits that are useful in the treatment of cancer. One kit of the invention includes a sustained release vehicle containing an immunostimulatory nucleic acid and a container housing a cancer medicament and instructions for timing of administration of the immunostimulatory nucleic acid and the cancer medicament. A sustained release vehicle is used herein in accordance with its prior art meaning of any device which slowly releases the immunostimulatory nucleic acid.

Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The cancer medicament is housed in at least one container. The container may be a single container housing all of the cancer medicament together or it may be multiple containers or chambers housing individual dosages of the cancer medicament, such as a blister pack. The kit also has instructions for timing of administration of the cancer medicament. The instructions would direct the subject having cancer or at risk of cancer to take the cancer medicament at the appropriate time. For instance, the appropriate time for delivery of the medicament may be as the symptoms occur. Alternatively, the appropriate time for administration of the medicament may be on a routine schedule such as monthly or yearly.

Another kit of the invention includes at least one container housing an immunostimulatory nucleic acid and at least one container housing a cancer medicament and instructions for administering the compositions in effective amounts for inducing a synergistic response in the subject. The immunostimulatory nucleic acid and cancer medicament may be housed in single containers or in separate compartments or containers, such as single dose compartments. The instructions in the kit direct the subject to take the immunostimulatory nucleic acid and the cancer medicament in amounts which will produce a synergistic response. The drugs may be administered simultaneously or separately as long as they are administered close enough in time to produce a synergistic response.

The pharmaceutical compositions of the invention contain an effective amount of an immunostimulatory nucleic acid and optionally cancer medicament and/or other therapeutic agents optionally included in a pharmaceutically-acceptable carrier. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The immunostimulatory nucleic acids and cancer medicament may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Another suitable compound for sustained release delivery is GELFOAM, a commercially available product consisting of modified collagen fibers.

Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The immunostimulatory nucleic acid compositions and the cancer medicament compositions can be administered on fixed schedules or in different temporal relationships to one another. The various combinations have many advantages over the prior art methods of treating cancer, particularly with regard to increased specific cancer toxicity and decreased non-specific toxicity to normal tissues.

Cancer medicaments and immunostimulatory nucleic acids can be administered by any ordinary route for administering medications. Depending upon the type of cancer to be treated, cancer medicaments and the nucleic acids of the invention may be inhaled, ingested or administered by systemic routes. Systemic routes include oral and parenteral. Inhaled medications are preferred in some embodiments because of the direct delivery to the lung, particularly in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Preferred routes of administration include but are not limited to oral, parenteral, intramuscular, intranasal, intratracheal, intrathecal, intravenous, inhalation, ocular, vaginal, and rectal.

For use in therapy, an effective amount of the immunostimulatory nucleic acid can be administered to a subject by any mode that delivers the nucleic acid to the affected organ or tissue, or alternatively to the immune system. “Administering” the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal.

For oral administration, the compounds (i.e., immunostimulatory nucleic acids, cancer medicament, and the other therapeutic agent, such as adjuvants) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic, such as the immunostimulatory capacity of the nucleic acids (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

In still other embodiments of the invention, the immunostimulatory nucleic acids are provided in the intravenous solutions, bags and/or tubing used to deliver transfusions into cancer patients. The immunostimulatory nucleic acids may be introduced into an intravenous solution which is administered to the subject prior to receiving the transfusion, or it may be introduced into the blood transfusion itself (i.e., the suspension of red blood cells or platelets). Alternatively, the intravenous bags and tubing may be themselves be coated on their internal surfaces with immunostimulatory nucleic acids, or they may be impregnated with immunostimulatory nucleic acids during manufacture. Methods for manufacture of intravenous systems for the delivery of biologically active materials are known in the art. Examples include those described in U.S. Pat. Nos. 4,973,307, and 5,250,028, issued to Alza, Corp. It is to be understood that the invention intends to embrace the use of immunostimulatory nucleic acids in reducing the side effects of blood transfusions (particularly the allergic reactions which commonly occur in subjects receiving such transfusions) in any subject in need of a blood transfusion, and not just cancer subjects.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for treating a subject having, or at risk of developing, a cancer, comprising:
administering to a subject in need of such treatment a poly-G nucleic acid and a cancer medicament in an effective amount to treat the cancer or to reduce the risk of developing the cancer,
wherein the poly-G nucleic acid is not conjugated to the cancer medicament.

2. The method of claim 1, wherein the cancer medicament is selected from the group consisting of a chemotherapeutic agent, an immunotherapeutic agent, and a cancer vaccine.

3. The method of claim 2, wherein the chemotherapeutic agent is selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Placlitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

4. The method of claim 2, wherein the immunotherapeutic agent is selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA.

5. The method of claim 2, wherein the cancer vaccine is selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys.

6. The method of claim 1, wherein the cancer medicament is a hormone therapy.

7. The method of claim 1, wherein the cancer medicament is taxol.

8. The method of claim 1, further comprising administering interferon-α to the subject.

9. The method of claim 1, wherein the cancer is selected from the group consisting of bone cancer, brain and CNS cancer, connective tissue cancer, esophageal cancer, eye cancer, Hodgkin's lymphoma, larynx cancer, oral cavity cancer, skin cancer, and testicular cancer.

10. The method of claim 1, wherein the immunostimulatory nucleic acid has a modified backbone.

11. The method of claim 10, wherein the modified backbone is a phosphorothioate modified backbone.

12. A method for treating a subject having or at risk of developing a cancer, comprising:
administering to a subject in need of such treatment, an immunostimulatory nucleic acid having a modified backbone and a cancer medicament selected from the group consisting of an immunotherapeutic agent, a cancer vaccine and a hormone therapy,
wherein the immunostimulatory nucleic acid is free of a CpG motif, and a T-rich motif.

13. The method of claim 12, wherein the immunostimulatory nucleic acid is a poly-G nucleic acid.

14. The method of claim 13, wherein the poly-G nucleic acid is not conjugated to the cancer medicament.

15. The method of claim 12, wherein the cancer medicament is taxol.

16. The method of claim 12, further comprising administering interferon-α to the subject.

17. The method of claim 12, further comprising administering a cancer antigen to the subject.

18.-20. (canceled)

21. A method for preventing an allergic reaction in a subject receiving a blood transfusion, comprising
administering to a subject receiving a blood transfusion an immunostimulatory nucleic acid in an effective amount to prevent an allergic reaction to the blood transfusion.

22.-30. (canceled)

31. A method for treating a subject having or at risk of developing cancer, comprising
administering to a subject in need of such treatment an immunostimulatory nucleic acid selected from the group consisting of a CpG nucleic acid and a non-CpG nucleic acid, and a cancer medicament that is a hormone therapy.

32.-35. (canceled)

36. A device for delivering an immunostimulatory nucleic acid to a subject receiving an intravenous injection, comprising
an intravenous device selected from the group consisting of an intravenous bag and an intravenous tube, and an immunostimulatory nucleic acid,
wherein the immunostimulatory nucleic acid is coated on an internal surface of the intravenous device or is embedded within the intravenous device.

37. A method for treating a subject having cancer comprising administering to the subject an immunostimulatory nucleic acid in a colloidal dispersion system, wherein the immunostimulatory nucleic acid is 8-100 nucleotides in length, and wherein the subject is receiving or has received radiation.

38. The method of claim 37, wherein the immunostimulatory nucleic acid is a CpG nucleic acid.

39. The method of claim 38, wherein the colloidal dispersion system is a liposome.

40. The method of claim 39, wherein the immunostimulatory nucleic acid is administered following radiation.

41. A method comprising administering an immunostimulatory nucleic acid in an implant to a subject that is receiving radiation, wherein the nucleic acid is 8-100 nucleotides in length.

42. The method of claim 41, wherein the immunostimulatory nucleic acid is a CpG nucleic acid.

43. The method of claim 41, further comprising stimulating an immune response in the subject.

44. The method of claim 41, wherein the subject has cancer.

45. The method of claim 44, wherein the cancer is basal cell carcinoma, biliary tract cancer, bladder cancer, bone cancer, brain cancer, CNS cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm; kidney cancer, larynx cancer, leukemia, liver cancer, small cell lung cancer, non-small cell lung cancer, lymphoma, Hodgkin's lymphoma, Non-Hodgkin's lymphoma, melanoma, myeloma, neuroblastoma, oral cavity cancer, ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system, as well as other carcinomas and sarcomas.

46. The method of claim 41, wherein the nucleic acid is administered following radiation.

47. The method of claim 41, wherein the nucleic acid is synthetic

48. A kit comprising an immunostimulatory nucleic acid and a chemical/physical vector, wherein the nucleic acid is 8-100 nucleotides in length.

49. The kit of claim 48, wherein the chemical/physical vector is an implant.

50. The kit of claim 48, further comprising a cancer medicament, wherein the cancer medicament is a chemotherapy.

51. A method of treating a subject with cancer comprising: administering at least one ligand for a pattern recognition receptor and a delivery vehicle; in conjunction with at least one cancer therapy wherein said method elicits a response in a subject disposed of cancer.

52. The method of claim 51, wherein said cancer therapy comprises at least one therapy consisting of hyperthermia therapy, radiation therapy, chemotherapy, photodynamic therapy (PDT), surgery, ultrasound, and focused ultrasound.

53. The method of claim 52, wherein radiation therapy is introduced first.

54. The method of claim 51, wherein the pattern recognition receptor ligand comprises a nucleic acid molecule.

55. The method of claim 51, wherein the delivery vehicle comprises a liposome.

56. The method of claim 51, wherein the delivery vehicle comprises a non-liposomal delivery vehicle.

57. A method comprising: coating a medical device with a composition comprising at least one ligand for a pattern recognition molecule receptor; and a delivery vehicle.

58. The method of claim 57, wherein the medical device comprises an implanted device.

59. The method of claim 58, wherein the implanted device consists of at least one of the following devices consisting of a catheter, a stent, a mesh repair material, a Dacron vascular prothesis, a orthopedic metallic plate, a rod and a screws.

60. The method of claim 57, wherein the delivery device comprises a sustained release particle and a delivery vehicle.

61. The method of claim 60, wherein the delivery vehicle comprises a liposome.

62. A method comprising: administering a composition comprising at least one ligand for a pattern recognition molecule receptor; a delivery device; and radiation therapy to a subject.

63. The method of claim 62, wherein a ligand for a pattern recognition molecule receptor comprises a ligand for a signaling pattern recognition receptor.

64. The method of claim 62, wherein a ligand for a pattern recognition molecule receptor comprises a ligand for an pattern recognition receptor.

65. The method of claim 62, further comprising augmenting an immune response in said subject.

66. The method of claim 65, wherein augmenting an immune response comprises augmenting an immune response in a subject disposed of cancer.

67. The method of claim 66, wherein cancer comprises at least one cancer selected from the group consisting of lung cancer, skin cancer, liver cancer, bone marrow cancer, brain cancer, renal cell cancer, ovarian cancer, breast cancer, prostate cancer, cancers of mesenchymal tissues, lymphoma and colon cancer.

68. The method of claim 62, wherein radiation therapy is introduced first.

69. The method of claim 62, wherein the ligand comprises a synthetic compound capable of binding a pattern recognition receptor.

70. A kit comprising: a delivery container; a delivery device; at least one ligand for a pattern recognition receptor; and plus or minus an antigen; wherein said ligand is capable of eliciting an immune response in a subject.

71. The kit of claim 70, further comprising one or more chemotherapy agents.
